(19) United States
(12) Patent Application Publication
(10) Pub. No.: US 2012/0278944 A1
(43) Pub. Date: Nov. 1, 2012

(54) ALTERED SENESCENCE FOR IMPROVED FORAGE QUALITY

(76) Inventors: Zeng-Yu Wang, Ardmore, OK (US); Chuanen Zhou, Ardmore, OK (US); Lu Han, Ardmore, OK (US)

(21) Appl. No.: 13/457,470

(22) Filed: Apr. 26, 2012

Related U.S. Application Data
(60) Provisional application No. 61/480,363, filed on Apr. 28, 2011.

(51) Int. Cl. A01H 5/00; C12N 5/04; A01H 5/06; A01H 1/00; A01H 5/02; C07H 21/00; C12N 5/10; A01H 5/10; A01H 5/12

(52) U.S. Cl. 800/278; 800/298; 435/410; 536/24.1; 435/419

(57) ABSTRACT

The invention provides methods and compositions for enhancing agronomic properties in plants through modification of senescence. Nucleic acid constructs therefore are also described. Transgenic plants are also provided that exhibit enhanced agronomic properties. Plants described herein may be used, for example, as improved forage crops.

This application claims the priority of U.S. Provisional Application No. 61/480,363, filed on Apr. 28, 2011, the entire disclosure of which is incorporated herein by reference.

### BACKGROUND OF THE INVENTION

1. Field of the Invention

The present invention relates generally to the field of agriculture and plant genetics. More particularly, it concerns genetically modified plants having enhanced agronomic properties.

2. Description of Related Art

Genetic modification of plants has, in combination with conventional breeding programs, led to significant increases in agricultural yield over the last decades. However, most genetically modified plants are selected for a single agronomic trait often by expression of a single enzyme coding sequence (e.g., enzymes that provide herbicide resistance). To date, there has been little progress in developing plants that comprise modified gene expression profiles and thereby exhibit a variety of characteristics that are of agronomic interest.

In particular, senescence of forage and turf is of special interest because the market value of these widely grown species is closely related to the visual appearance of their foliage. The most apparent phenomenon of plant senescence is leaf color change from green to yellow or red. Alfalfa (Medicago sativa) is the third largest economic crop and the most important forage legume in the United States with approximately 9.6 million hectares in production and an estimated value for alfalfa hay alone of $8 billion. Most alfalfa is used for hay production, and a key trait for evaluation of hay value at the market is the color of alfalfa, with green leaves reflecting premium quality. Senescence causes leaf yellowing and reduces market value.

### SUMMARY OF THE INVENTION

In one aspect, the invention provides a forage crop plant comprising down-regulated SGR gene function where the plant exhibits an enhanced agronomic property as a result of the down-regulated SGR gene function. In certain aspects, the plant comprises a DNA molecule capable of expressing a nucleic acid sequence complementary to all or a portion of a SGR messenger RNA (mRNA). In further aspects, the plant comprises a DNA molecule complementary to all or a portion of a SGR mRNA wherein the DNA molecule down-regulates the function of the SGR gene relative to a plant lacking the DNA molecule. In additional aspects, the plant may be further defined as transformed with a DNA molecule complementary to all or a portion of SEQ ID NO:6. In other aspects, the DNA molecule is operably linked to a developmentally-regulated, organelle-specific, inducible, tissue-specific, constitutive, cell-specific, seed specific, or germination-specific promoter. In one aspect, the plant is alfalfa.

In another aspect, the plant may comprise a mutation in a SGR gene relative to a wild-type plant of the same species, and the mutation may be a deletion, a point mutation or an insertion in the SGR gene and further be produced by irradiation, T-DNA insertion, transposon insertion or chemical mutagenesis.

In still another aspect, the invention provides a plant with a down-regulated SGR gene that has an enhanced agronomic property is selected from the group consisting of increased chlorophyll content, increased forage nutritional content, increased yield, and improved visual appearance compared to a plant in which the SGR gene is not down-regulated.

A transgenic plant of the invention may be a fertile R₀ transgenic plant and may be further defined as a progeny plant of any generation of a fertile R₀ transgenic plant, wherein the transgenic plant comprises the selected DNA. A seed or plant part of a transgenic plant of the invention is also provided, wherein the seed or plant part comprises the DNA molecule, and where the plant part is further defined a protoplast, cell, meristem, root, leaf, node, pistil, anther, flower, seed, embryo, stalk or petiole.

In still yet another aspect, the invention provides a plant comprises an SGR gene encoding a polypeptide comprising a sequence selected from the group consisting of SEQ ID NO:2; SEQ ID NO:4; SEQ ID NO:20; SEQ ID NO:22; SEQ ID NO:24; SEQ ID NO:26; SEQ ID NO:28; SEQ ID NO:30; SEQ ID NO:32; SEQ ID NO:34; SEQ ID NO:36; SEQ ID NO:37; SEQ ID NO:38; SEQ ID NO:39; SEQ ID NO:40; and SEQ ID NO:41.

In one aspect, the invention provides a nucleic acid molecule comprising a nucleic acid sequence selected from the group consisting of (a) a nucleic acid sequence that hybridizes to the sequence of SEQ ID NO:1; SEQ ID NO:3; SEQ ID NO:5; SEQ ID NO:19; SEQ ID NO:21; SEQ ID NO:23; SEQ ID NO:25; SEQ ID NO:27; SEQ ID NO:29; SEQ ID NO:31; SEQ ID NO:33; or SEQ ID NO:35 under conditions of 1×SSC and 65° C.; (b) a nucleic acid comprising SEQ ID NO:1; SEQ ID NO:3; SEQ ID NO:5; SEQ ID NO:19; SEQ ID NO:21; SEQ ID NO:23; SEQ ID NO:25; SEQ ID NO:27; SEQ ID NO:29; SEQ ID NO:31; SEQ ID NO:33; or SEQ ID NO:35 or a fragment thereof; and (c) a nucleic acid sequence exhibiting at least 80% sequence identity to SEQ ID NO:1; SEQ ID NO:3; SEQ ID NO:5; SEQ ID NO:19; SEQ ID NO:21; SEQ ID NO:23; SEQ ID NO:25; SEQ ID NO:27; SEQ ID NO:29; SEQ ID NO:31; SEQ ID NO:33; or SEQ ID NO:35; and (d) a fragment of at least 19 contiguous nucleotides of a nucleic acid sequence of SEQ ID NO:1; SEQ ID NO:3; SEQ ID NO:5; SEQ ID NO:19; SEQ ID NO:21; SEQ ID NO:23; SEQ ID NO:25; SEQ ID NO:27; SEQ ID NO:29; SEQ ID NO:31; SEQ ID NO:33; or SEQ ID NO:35, or the reverse complement thereof, wherein the presence in a plant of a double stranded ribonucleotide sequence comprising at least one strand that is complementary to said fragment down-regulates SGR gene function in the plant; wherein the nucleic acid sequence is operably linked to a heterologous promoter sequence. In still another aspect, the heterologous promoter sequence is a developmentally-regulated, organelle-specific, inducible, tissue-specific, constitutive, cell-specific, seed specific, or germination-specific promoter. In one aspect, a transgenic plant or plant part comprises the nucleic acid molecule

In a further aspect, the nucleic acid molecule comprises a nucleic acid sequence exhibiting at least 85%, 90%, 95%, 96%, 97%, 98% or 99% sequence identity to SEQ ID NO:1; SEQ ID NO:3; SEQ ID NO:5; SEQ ID NO:19; SEQ ID NO:21; SEQ ID NO:23; SEQ ID NO:25; SEQ ID NO:27; SEQ ID NO:29; SEQ ID NO:31; SEQ ID NO:33; or SEQ ID NO:35.

In still another aspect, the present invention provides a method for producing forage comprising (a) obtaining a forage crop plant comprising down-regulated SGR gene function where the plant exhibits an enhanced agronomic property as a result of the down-regulated SGR gene function, and (b) collecting biomass for forage.

In yet another aspect, the present invention provides a method of conferring at least a first altered agronomic property to a plant comprising down-regulating SGR gene function in the plant relative to a plant in which SGR gene function is not down-regulated. In certain aspects, the altered agronomic property is selected from the group consisting of increased nutritional content, increased chlorophyll content, increased yield, and improved visual appearance.

Embodiments discussed in the context of methods and/or compositions of the invention may be employed with respect to any other method or composition described herein. Thus, an embodiment pertaining to one method or composition may be applied to other methods and compositions of the invention as well.

As used herein the terms “encode” or “encoding” with reference to a nucleic acid are used to make the invention readily understandable by the skilled artisan however these terms may be used interchangeably with “comprise” or “comprising” respectively.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the phenotypic characterization of the Tnt1-tagged Medicago truncatula mutant line NF2089. (A) Natural senescence of leaves at the bottom of wild-type (left) (SEQ ID NO:1) and NF2089 (right) after 60-days of vegetative growth. (B) Natural senescence of wild-type (left) and NF2089 (right) plants. (C) Color change in mature anther and central carpels of wild-type (left) and NF2089 (right). (D) Mature pods of wild-type (left) and NF2089 (right). (E) Mature seeds of wild-type (left) and NF2089 (right).

FIG. 2 shows the stay-green phenotype and chlorophyll characterization of the NF2089 mutant during dark-induced senescence. (A) Color change in detached leaves of wild-type and NF2089 during dark-induced senescence. (B) Chlorophyll degradation during dark incubation. (C) Changes in contents of Chl a and Chl b during senescence. Closed circles, Chl a. Open circles, Chl b. (D) Change in Fᵥ/Fₘ values during dark incubation. Error bars indicate SE (n=3). 0, 5, 10 indicate days after dark treatment (DAD).

FIG. 3 shows the fluorescent intensity and ultra-structural observation of chloroplasts in wild-type and the NF2089 mutant. (A)-(D) Detection of auto-fluorescence of chlorophyll in mesophyll cells using confocal microscope. Wild-type (A, B) and NF2089 (C, D) at 0 DAD (A, C) and 10 DAD (B, D). (E)-(H) Ultra-structure of chloroplasts observed using electron microscope. Wild-type (E, F) and NF2089 (G, H) at 0 DAD (E, G) and 10 DAD (F, H). DAD: days after dark treatment. Arrow heads indicate the thick and consecutive granum stacks. g, grana stack. p, plastoglobule. s, starch granule. Bar: 10 μm in (A)-(D). Bar: 500 nm in (E)-(H).

FIG. 4 illustrates the molecular cloning and expression pattern of the MtSGR gene. (A) Diagram of the MtSGR gene (1734 bp) (SEQ ID NO:1) structure showing the four exons (block), three introns (line) and the positions of Tnt1 insertions. (B) PCR amplification of MtSGR from genomic DNA of wild-type and NF2089 showing the presence of a 5.3 Kb Tnt1 insertion in the mutant. (C) RT-PCR showed that transcription of MtSGR was interrupted in NF2089. (D) Expression of MtSGR in leaves of wild-type 0, 5, 10 days after dark treatment (DAD) analyzed by RT-PCR. (E) Expression pattern of MtSGR in different organs and development stages in wild-type. Bars: 200 bp.

FIG. 5 illustrates the statistically enriched Gene Ontology terms level 2 in the NF2089 mutant. The plot was produced using the online tool WEGO. (A) Analysis of GO terms in up-regulated genes. (B) Analysis of GO terms in down-regulated genes.

FIG. 6 is a phylogenetic analysis of SGRs among diversified plant species. (A) Phylogenetic tree was constructed with deduced SGR amino acid sequences. The sequences include M. truncatula (MtSGR) (SEQ ID NO:2), alfalfa (MsSGR) (SEQ ID NO:4), pea (PsSGR) (SEQ ID NO:28), soybean (GmSGR1 and GmSGR2) (SEQ ID NO:24 and SEQ ID NO:26), tobacco (NtSGR) (SEQ ID NO:37), pepper (CaSGR) (SEQ ID NO:30), tomato (SlSGR) (SEQ ID NO:32), Arabidopsis (AtNYE1 and AtNYE2) (SEQ ID NO:20 and SEQ ID NO:22), rice (OsSGR) (SEQ ID NO:34), sorghum (SbSGR) (SEQ ID NO:36), corn (ZmSGR1 and ZmSGR2) (SEQ ID NO:38 and SEQ ID NO:39), and moss (PpSGR1 and PpSGR2) (SEQ ID NO:40 and SEQ ID NO:41). (B) Alignment of predicted amino acid sequences of these putative SGR orthologs. Asterisk indicates the insertion site of the Tnt1 retrotransposon, revealing this amino acid is conserved. Conserved residues across all the species are shaded black; residues that are conserved in ten to fifteen species are shaded gray.

FIG. 7 is the expression pattern of the MsSGR gene (SEQ ID NO:3) in alfalfa nodule. Accumulation of MsSGR transcripts in NO₃-induced nodule senescence and natural nodule senescence in alfalfa. dpi: days after rhizobial inoculation. Error bars indicate SE (n=3).

FIG. 8 illustrates the molecular characterization of alfalfa MsSGR-RNAi transgenic lines. (A) PCR analysis of regenerated alfalfa plants comprising the 543 bp SGR fragment (SEQ ID NO:5) together with the positive control (pANDA35HK plasmid), negative control (wild-type) and empty vector control (CTRL). The size of DNA fragments: 699 bp for nptII, 375 bp for hph, and 633 bp for gus linker. (B) Quantitative real time RT-PCR analysis of MsSGR gene expression in transgenic lines. All the values are normalized using the empty vector control. Error bars indicate SE (n=3). (C) Southern blot analysis of Kpn I digested genomic DNA from leaves of wild-type and MsSGR-RNAi transgenic lines. The DNA was probed with 699 bp of gus linker fragment from the pANDA35HK vector. (D) A schematic map of the RNAi construct.

FIG. 9 illustrates the dark incubation of MsSGR-RNAi transgenic lines. (A) Detached leaves of wild-type, empty vector control (CTRL) and MsSGR-RNAi transgenic lines in the dark. (B) Living plants of wild-type and a transgenic line (SGRi-39) in the dark. 0, 5, 10, 15, 20 indicate days after dark treatment (DAD).

FIG. 10 shows naturally fallen leaves of wild-type (A), empty vector control (B, C) and transgenic alfalfa lines SGRi-10 (D), SGRi-17 (E), SGRi-21 (F), SGRi-29 (G) and SGRi-39 (H). Transgenic leaves showed more greenish color.

FIG. 11 is a characterization of chloroplasts of control and alfalfa MsSGR-RNAi transgenic lines during dark-induced senescence. (A)-(F), auto-fluorescence intensity during senescence of control (A-C) and the transgenic line SGRi-39 (D-F). (G)-(L) Ultra-structures of chloroplasts of the control (G-I) and SGRi-39 (J-L). An arrow indicates reduced thylakoid membranes. Arrow heads show the thick and consecutive granum stacks. g, grana stack. p, plastoglobule. s, starch granule. 0, 10, 20 indicate days after dark treatment (DAD). Bar: 10 μm in (A)-(F); 500 nm in (G)-(L).

FIG. 12 shows measurements of chlorophyll content and Fv/Fm values in control and independent MsSGR-RNAi transgenic alfalfa lines during dark incubation. (A) Change of chlorophyll content during dark incubation. (B) Chl a/b ratio of the same samples as in (A). (C) Fᵥ/Fₘ value. CTRL: control plants. 0, 5, 10, 15, 20 indicate days after dark treatment (DAD). Error bars indicate SE (n=3).

FIG. 13 illustrates the phenotypic appearance of control and MsSGR-RNAi transgenic alfalfa under natural drying conditions. (A) Control plants. (B) SGRi-39 transgenic line. 0 d, 5 d, 10 d, 15 d, 20 d indicate days after drying of the material.

FIG. 14 is an evaluation of forage quality of transgenic alfalfa lines. Eight parameters related to forage nutritive quality, including crude protein (CP), dry matter (DM), acid detergent fiber (ADF), neutral detergent fiber (NDF), in vitro true dry matter digestibility (IVTDMD), total digestible nutrients (TDN), concentrations of phosphorus (P) and magnesium (Mg) were measured. Error bars indicate SE (n=3).

### BRIEF DESCRIPTION OF THE SEQUENCES

SEQ ID NO:1 is a M. truncatula STAY GREEN (SGR) DNA sequence; NCBI Accession No. HQ849484.

SEQ ID NO:2 is a M. truncatula SGR amino acid sequence encoded by the polynucleotide sequence of SEQ ID NO:1.

SEQ ID NO:3 is a M. sativa SGR nucleotide sequence; NCBI Accession No. HQ849485.

SEQ ID NO:4 is a M. sativa SGR amino acid sequence encoded by the polynucleotide of SEQ ID NO:3.

SEQ ID NO:5 is a M. sativa MsSGR DNA fragment.

SEQ ID NO:6-18 are PCR primers.

SEQ ID NO:19 is a polynucleotide encoding NYE1 from Arabidopsis thaliana.

SEQ ID NO:20 is the NYE1 amino acid sequence from A. thaliana encoded by the polynucleotide sequence of SEQ ID NO:19.

SEQ ID NO:21 is a polynucleotide encoding NYE2 from A. thaliana.

SEQ ID NO:22 is the NYE2 amino acid sequence from A. thaliana encoded by the polynucleotide sequence of SEQ ID NO:21.

SEQ ID NO:23 is a polynucleotide encoding SGR1 from soybean (Glycine max); NCBI Accession No. AY850141.

SEQ ID NO:24 is the SGR1 amino acid sequence from Glycine max encoded by the polynucleotide sequence of SEQ ID NO:23.

SEQ ID NO:25 is a polynucleotide encoding SGR2 from soybean (Glycine max); NCBI Accession No. AY850142.

SEQ ID NO:26 is the SGR2 amino acid sequence from Glycine max encoded by the polynucleotide sequence of SEQ ID NO: 25.

SEQ ID NO:27 is a polynucleotide encoding SGR from pea (Pisum sativum); NCBI Accession No. AB303331.

SEQ ID NO:28 is the SGR amino acid sequence from P. sativum encoded by the polynucleotide sequence of SEQ ID NO:27.

SEQ ID NO:29 is a polynucleotide encoding SGR from pepper (Capsicum annuum); NCBI Accession No. EU414631.

SEQ ID NO:30 is the SGR amino acid sequence from C. annuum encoded by the polynucleotide sequence of SEQ ID NO:29.

SEQ ID NO:31 is a polynucleotide encoding SGR from tomato (Solanum lycopersicum); NCBI Accession No. EU414632.

SEQ ID NO:32 is the SGR amino acid sequence from S. lycopersicum encoded by the polynucleotide sequence of SEQ ID NO:21.

SEQ ID NO:33 is a polynucleotide encoding SGR from rice (Oryza sativa); NCBI Accession No. AY850134.

SEQ ID NO:34 is the SGR amino acid sequence from O. sativa encoded by the polynucleotide sequence of SEQ ID NO:33.

SEQ ID NO:35 is a polynucleotide encoding SGR from sorghum (Sorghum bicolor); NCBI Accession No. AY850140.

SEQ ID NO:36 is the SGR amino acid sequence from S. bicolor encoded by the polynucleotide sequence of SEQ ID NO:35.

SEQ ID NO:37 is a SGR amino acid sequence from tobacco (Nicotiana tabacum); NCBI Accession No. ABY19382.

SEQ ID NO:38 is a SGR amino acid sequence, SGR1, from corn (Zea mays); NCBI Accession No. AAW82956.

SEQ ID NO:39 is a SGR amino acid sequence, SGR2, from corn (Z. mays); NCBI Accession No. NP_{—}001105771.

SEQ ID NO:40 is a predicted SGR amino acid sequence, SGR1, from moss (Physcomitrella patens); NCBI Accession No. EDQ70701.

SEQ ID NO:41 is a predicted SGR amino acid sequence, SGR2, from moss (P. patens); NCBI Accession No. EDQ62217.

### DESCRIPTION OF ILLUSTRATIVE EMBODIMENTS

The invention provides methods and compositions for improving forage quality such as by down-regulating the expression of a gene involved in senescence in a plant cell. In certain embodiments, the methods surprisingly give rise to enhanced agronomic properties.

In one aspect, the invention provides methods involved in down-regulating a Stay-Green (SGR) gene, surprisingly giving rise to an enhanced agronomic property such as improved quality of forage. The invention therefore provides in certain embodiments expression cassettes comprising a nucleotide sequence that down-regulates the SGR gene in a plant cell, operably linked to a promoter that directs expression of the nucleotide sequence in the plant cell. In some aspects, the SGR gene may be mutated to achieve down-regulation, including where the mutation may be made by deletion, a point mutation, an insertion, or is alternatively produced by irradiation or chemical mutagenesis.

The enhanced agronomic properties resulting from down-regulation of SGR gene activation in some aspects include altered chlorophyll content, increased forage nutritional content, increased yield, and improved visual appearance, such as greener, healthier appearance characteristic of a plant and biomass therefrom.

In one embodiment, SGR gene expression may be down-regulated by expressing in the cell a nucleotide sequence that suppresses expression. Suppression of expression of a gene may be accomplished by any method known in the art, for instance via RNAi-mediated suppression, among other approaches.

### I. DNA Molecules and Plant Transformation Constructs

Of particular interest are polynucleotide molecules wherein the polynucleotide molecules function in plants and have at least about 60% sequence identity, at least about 70% sequence identity, at least about 80% sequence identity, at least about 90% sequence identity, or even greater sequence identity, specifically including about 73%, 75%, 78%, 83%, 85%, 88%, 92%, 93%, 94%, 95%, 96%, 97%, 98%, or 99% or greater sequence identity with the nucleotide sequence of SEQ ID NO:1; SEQ ID NO:3; SEQ ID NO:5; SEQ ID NO:19; SEQ ID NO:21; SEQ ID NO:23; SEQ ID NO:25; SEQ ID NO:27; SEQ ID NO:29; SEQ ID NO:31; SEQ ID NO:33; or SEQ ID NO:35. In certain embodiments of the invention, nucleic acids hybridizing to SEQ ID NO:1; SEQ ID NO:3; SEQ ID NO:5; SEQ ID NO:19; SEQ ID NO:21; SEQ ID NO:23; SEQ ID NO:25; SEQ ID NO:27; SEQ ID NO:29; SEQ ID NO:31; SEQ ID NO:33; or SEQ ID NO:35 or a complement or reverse complement thereof, under stringent conditions, are provided. Such conditions are well known in the art, such as 1×SSC and 65° C. The invention further provides nucleic acid sequences that encode a sequence complementary to all or a part of an mRNA encoded by an SGR gene, as described herein and known in the art, wherein the expression of the sequences functions to down-regulate the gene. In certain embodiments of the invention, fragments or complements thereof of at least 21 contiguous nucleotides of a nucleic acid sequence of SEQ ID NO:1; SEQ ID NO:3; SEQ ID NO:5; SEQ ID NO:19; SEQ ID NO:21; SEQ ID NO:23; SEQ ID NO:25; SEQ ID NO:27; SEQ ID NO:29; SEQ ID NO:31; SEQ ID NO:33; or SEQ ID NO:35 are provided that at least partially inhibit senescence of a plant.

In a certain further embodiment, DNA constructs for plant transformation are provided. For example, a DNA construct can be for expression of an antisense RNA, siRNA or miRNA that down-regulates or up-regulates expression of SGR. Vectors used for plant transformation may include, for example, plasmids, cosmids, YACs (yeast artificial chromosomes), BACs (bacterial artificial chromosomes) or any other suitable cloning system, as well as fragments of DNA therefrom. Thus when the term “vector” or “expression vector” is used, all of the foregoing types of vectors, as well as nucleic acid sequences isolated therefrom, are included. It is contemplated that utilization of cloning systems with large insert capacities will allow introduction of large DNA sequences comprising more than one selected gene. Introduction of such sequences may be facilitated by use of bacterial or yeast artificial chromosomes (BACs or YACs, respectively), or even plant artificial chromosomes. For example, the use of BACs for Agrobacterium-mediated transformation was disclosed by Hamilton et al., (1996).

Particularly useful for transformation are expression cassettes which have been isolated from such vectors. DNA segments used for transforming plant cells will, of course, generally comprise the cDNA, gene or genes which one desires to introduce into and have expressed in the host cells. These DNA segments can further include structures such as promoters, enhancers, polylinkers, or even regulatory genes as desired. The DNA segment or gene chosen for cellular introduction will often encode a protein which will be expressed in the resultant recombinant cells resulting in a screenable or selectable trait and/or which will impart an improved phenotype to the resulting transgenic plant. However, this may not always be the case, and the present invention also encompasses transgenic plants incorporating non-expressed transgenes. Preferred components likely to be included with vectors used in the current invention are as follows.

A. Regulatory Elements

Exemplary promoters for expression of a nucleic acid sequence include plant promoters such as the CaMV 35S promoter (Odell et al., 1985), or others such as CaMV 19S (Lawton et al., 1987), nos (Ebert et al., 1987), Adh (Walker et al., 1987), sucrose synthase (Yang and Russell, 1990), α-tubulin, actin (Wang et al., 1992), cab (Sullivan et al., 1989), PEPCase (Hudspeth and Grula, 1989) or those associated with the R gene complex (Chandler et al., 1989). Tissue specific promoters such as root cell promoters (Conkling et al., 1990) and tissue specific enhancers (Fromm et al., 1986) are also contemplated to be useful, as are inducible promoters such as ABA- and turgor-inducible promoters.

The DNA sequence between the transcription initiation site and the start of the coding sequence, i.e., the untranslated leader sequence, can also influence gene expression. One may thus wish to employ a particular leader sequence with a transformation construct of the invention. Leader sequences include those which comprise sequences predicted to direct optimum expression of the attached gene, i.e., to include a preferred consensus leader sequence which may increase or maintain mRNA stability and prevent inappropriate initiation of translation. The choice of such sequences will be known to those of skill in the art in light of the present disclosure. Sequences that are derived from genes that are highly expressed in plants may be particularly useful.

It is contemplated that vectors for use in accordance with the present invention may be constructed to include an ocs enhancer element. This element was first identified as a 16 bp palindromic enhancer from the octopine synthase (ocs) gene of Agrobacterium (Ellis et al., 1987), and is present in at least 10 other promoters (Bouchez et al., 1989). The use of an enhancer element, such as the ocs element and particularly multiple copies of the element, may act to increase the level of transcription from adjacent promoters when applied in the context of plant transformation.

It is envisioned that SGR coding sequences (or complements thereof) may be introduced under the control of novel promoters or enhancers, etc., or homologous or tissue specific promoters or control elements. Vectors for use in tissue-specific targeting of genes in transgenic plants may include tissue-specific promoters and may also include other tissue-specific control elements such as enhancer sequences. Promoters that direct specific or enhanced expression in certain plant tissues will be known to those of skill in the art in light of the present disclosure. These include, for example, the rbcS promoter, specific for green tissue; the ocs, nos and mas promoters.

B. Terminators

Transformation constructs prepared in accordance with the invention will typically include a 3′ end DNA sequence that acts as a signal to terminate transcription and allow for the poly-adenylation of the mRNA produced by coding sequences operably linked to a promoter. It is envisioned that the native terminator of a SGR coding sequence can be used. Alternatively, a heterologous 3′ end may enhance the expression of sense or antisense SGR coding sequences and fragments. Examples of terminators include those from the nopaline synthase gene of Agrobacterium tumefaciens (nos 3′ end) (Bevan et al., 1983), the terminator for the T7 transcript from the octopine synthase gene of Agrobacterium tumefaciens, and the 3′ end of the protease inhibitor I or II genes from potato or tomato. Regulatory elements such as an Adh intron (Callis et al., 1987), sucrose synthase intron (Vasil et al., 1989) or TMV omega element (Gallie et al., 1989), may further be included where desired.

C. Transit or Signal Peptides

Sequences that are joined to the coding sequence of an expressed gene, which are removed post-translationally from the initial translation product and that facilitate the transport of the protein into or through intracellular or extracellular membranes, are termed transit (usually into vacuoles, vesicles, plastids and other intracellular organelles) and signal sequences (usually to the endoplasmic reticulum, golgi apparatus and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

D. Marker Genes

By employing a selectable or screenable marker protein, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Of course, many examples of suitable marker proteins are known to the art and can be employed in the practice of the invention.

Included within the terms “selectable” or “screenable” markers also are genes which encode a “secretable marker” whose secretion can be detected as a means of identifying or selecting for transformed cells. Examples include markers which are secretable antigens that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extensin or tobacco PR-S).

Many selectable marker coding regions are known and could be used with the present invention including, but not limited to, neo (Potrykus et al., 1985), which provides kanamycin resistance and can be selected for using kanamycin, G418, paromomycin, etc.; bar, which confers bialaphos or phosphinothricin resistance; a mutant EPSP synthase protein (Hinchee et al., 1988) conferring glyphosate resistance; a nitrilase such as bxn from Klebsiella ozaenae which confers resistance to bromoxynil (Stalker et al., 1988); a mutant acetolactate synthase (ALS) which confers resistance to imidazolinone, sulfonylurea or other ALS inhibiting chemicals (European Patent Application 154204, 1985); a methotrexate resistant DHFR (Thillet et al., 1988), a dalapon dehalogenase that confers resistance to the herbicide dalapon; or a mutated anthranilate synthase that confers resistance to 5-methyl tryptophan.

Screenable markers that may be employed include a β-glucuronidase (GUS) or uidA gene which encodes an enzyme for which various chromogenic substrates are known, a luciferase (lux) gene (Ow et al., 1986), which allows for bioluminescence detection; or a gene encoding for green fluorescent protein (Sheen et al., 1995; Haseloff et al., 1997; Reichel et al., 1996; Tian et al., 1997; WO 97/41228). The gene that encodes green fluorescent protein (GFP) is also contemplated as a particularly useful reporter gene (Sheen et al., 1995; Haseloff et al., 1997; Reichel et al., 1996; Tian et al., 1997; WO 97/41228). Expression of green fluorescent protein may be visualized in a cell or plant as fluorescence following illumination by particular wavelengths of light.

### II. Antisense and RNAi Constructs

Antisense and RNAi treatments represent one way of altering agronomic characteristics in accordance with the invention such as by down regulation of SGR. In particular, constructs comprising a SGR coding sequence, including fragments thereof, in antisense orientation, or combinations of sense and antisense orientation, may be used to decrease or effectively eliminate the expression of a SGR gene in a plant and to alter agronomic characteristics. Accordingly, this may be used to partially or completely “knock-out” the function of a SGR or homologous sequences thereof.

Techniques for RNAi suppression are well known in the art and are described in, for example, Lehner et al., (2004) and Downward (2004). The technique is based on the fact that double stranded RNA is capable of directing the degradation of messenger RNA with sequence complementary to one or the other strand (Fire et al., 1998). Therefore, by expression of a particular coding sequence in sense and antisense orientation, either as a fragment or longer portion of the corresponding coding sequence, the expression of that corresponding sequence can be down-regulated.

Antisense, and in some aspects RNAi, methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense oligonucleotides, when introduced into a target cell, specifically bind to their target polynucleotide and interfere with transcription, RNA processing, transport, translation and/or stability. Antisense and RNAi constructs, or DNA encoding such RNAs, may be employed to inhibit gene transcription or translation or both within a host cell, either in vitro or in vivo, such as within a host plant cell. In certain embodiments of the invention, such an oligonucleotide may comprise any unique portion of a nucleic acid sequence provided herein. In certain embodiments of the invention, such a sequence comprises at least 17, 18, 19, 20, 21, 25, 30, 50, 75 or 100 or more contiguous nucleic acids of the nucleic acid sequence of a SGR gene, and/or complements thereof, which may be in sense and/or antisense orientation. By including sequences in both sense and antisense orientation, increased suppression of the corresponding coding sequence may be achieved.

Constructs may be designed that are complementary to all or part of the promoter and other control regions, exons, introns or even exon-intron boundaries of a gene. It is contemplated that the most effective constructs may include regions complementary to intron/exon splice junctions. Thus, it is proposed that one embodiment includes a construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have few base mismatches. For example, sequences of eighteen bases in length may be termed complementary when they have complementary nucleotides at sixteen or seventeen positions. Naturally, sequences which are completely complementary will be sequences which are entirely complementary throughout their entire length and have no base mismatches. Other sequences with lower degrees of homology also are contemplated. For example, an RNAi or antisense construct which has limited regions of high homology, but also contains a non-homologous region (e.g., ribozyme; see above) could be designed. Methods for selection and design of sequences that generate RNAi are well known in the art (e.g., Reynolds, 2004). These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence. Constructs useful for generating RNAi may also comprise concatemers of sub-sequences that display gene regulating activity.

### III. Methods for Genetic Transformation

Suitable methods for transformation of plant or other cells for use with the current invention are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253, specifically incorporated herein by reference in its entirety), by agitation with silicon carbide fibers (Kaeppler et al., 1990; U.S. Pat. No. 5,302,523, specifically incorporated herein by reference in its entirety; and U.S. Pat. No. 5,464,765, specifically incorporated herein by reference in its entirety), by Agrobacterium-mediated transformation (U.S. Pat. No. 5,591,616 and U.S. Pat. No. 5,563,055; both specifically incorporated herein by reference) and by acceleration of DNA coated particles (U.S. Pat. No. 5,550,318; U.S. Pat. No. 5,538,877; and U.S. Pat. No. 5,538,880; each specifically incorporated herein by reference in its entirety), etc. Through the application of techniques such as these, the cells of virtually any plant species, including biofuel crop species, may be stably transformed, and these cells developed into transgenic plants.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et al., (1985), Rogers et al., (1987) and U.S. Pat. No. 5,563,055, specifically incorporated herein by reference in its entirety. Somleva et al., (2002) describe the creation of approximately 600 transgenic switchgrass plants carrying a bar gene and a uidA gene (beta-glucuronidase) under control of a maize ubiquitin promoter and rice actin promoter respectively. Both genes were expressed in the primary transformants and could be inherited and expressed in subsequent generations. Addition of 50 to 200 μM acetosyringone to the inoculation medium increased the frequency of transgenic switchgrass plants recovered.

Another widely applicable method for delivering transforming DNA segments to plant cells is microprojectile bombardment (U.S. Pat. No. 5,550,318; U.S. Pat. No. 5,538,880; U.S. Pat. No. 5,610,042; and PCT Application WO 94/09699; each of which is specifically incorporated herein by reference in its entirety). In this method, particles may be coated with nucleic acids and delivered into cells by a propelling force. Exemplary particles include those comprised of tungsten, platinum, and often, gold.

Examples of species for which have been transformed by microprojectile bombardment include monocot species such as maize (PCT Application WO 95/06128), barley (Ritala et al., 1994; Hensgens et al., 1993), wheat (U.S. Pat. No. 5,563,055, specifically incorporated herein by reference in its entirety), rice (Hensgens et al., 1993), oat (Torbet et al., 1995; Torbet et al., 1998), rye (Hensgens et al., 1993), sugarcane (Bower et al., 1992), and sorghum (Casa et al., 1993; Hagio et al., 1991); as well as a number of dicots including tobacco (Tomes et al., 1990; Buising and Benbow, 1994), soybean (U.S. Pat. No. 5,322,783, specifically incorporated herein by reference in its entirety), sunflower (Knittel et al., 1994), peanut (Singsit et al., 1997), cotton (McCabe and Martinell, 1993), tomato (VanEck et al., 1995), and legumes in general (U.S. Pat. No. 5,563,055, specifically incorporated herein by reference in its entirety).

Richards et al., (2001) describe the creation of transgenic switchgrass plants using particle bombardment. Callus was bombarded with a plasmid carrying a sgfp (green fluorescent protein) gene and a bar (bialaphos and Basta tolerance) gene under control of a rice actin promoter and maize ubiquitin promoter respectively. Plants regenerated from bombarded callus were Basta tolerant and expressed GFP. These primary transformants were then crossed with non-transgenic control plants, and Basta tolerance was observed in progeny plants, demonstrating inheritance of the bar gene.

Tissue cultures may be used in certain transformation techniques for the preparation of cells for transformation and for the regeneration of plants therefrom. Maintenance of tissue cultures requires use of media and controlled environments. “Media” refers to the numerous nutrient mixtures that are used to grow cells in vitro, that is, outside of the intact living organism. The medium usually is a suspension of various categories of ingredients (salts, amino acids, growth regulators, sugars, buffers) that are required for growth of most cell types. However, each specific cell type requires a specific range of ingredient proportions for growth, and an even more specific range of formulas for optimum growth. Rate of cell growth also will vary among cultures initiated with the array of media that permit growth of that cell type.

Nutrient media is prepared as a liquid, but this may be solidified by adding the liquid to materials capable of providing a solid support. Agar is most commonly used for this purpose. BACTOAGAR, GELRITE, and GELGRO are specific types of solid support that are suitable for growth of plant cells in tissue culture.

Some cell types will grow and divide either in liquid suspension or on solid media. As disclosed herein, plant cells will grow in suspension or on solid medium, but regeneration of plants from suspension cultures typically requires transfer from liquid to solid media at some point in development. The type and extent of differentiation of cells in culture will be affected not only by the type of media used and by the environment, for example, pH, but also by whether media is solid or liquid.

Tissue that can be grown in a culture includes meristem cells, Type I, Type II, and Type III callus, immature embryos and gametic cells such as microspores, pollen, sperm and egg cells. Type I, Type II, and Type III callus may be initiated from tissue sources including, but not limited to, immature embryos, seedling apical meristems, root, leaf, microspores and the like. Those cells which are capable of proliferating as callus also are recipient cells for genetic transformation.

### IV. Production and Characterization of Stably Transformed Plants

After effecting delivery of exogenous DNA to recipient cells, the next steps generally concern identifying the transformed cells for further culturing and plant regeneration. In order to improve the ability to identify transformants, one may desire to employ a selectable or screenable marker gene with a transformation vector prepared in accordance with the invention. In this case, one would then generally assay the potentially transformed cell population by exposing the cells to a selective agent or agents, or one would screen the cells for the desired marker gene trait.

A. Selection

It is believed that DNA is introduced into only a small percentage of target cells in any one study. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Resistance to the aminoglycoside antibiotics is conferred by aminoglycoside phosphotransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA.

B. Regeneration and Seed Production

Cells that survive the exposure to the selective agent, or cells that have been scored positive in a screening assay, may be cultured in media that supports regeneration of plants. In an exemplary embodiment, MS and N6 media may be modified by including further substances such as growth regulators. One such growth regulator is dicamba or 2,4-D. However, other growth regulators may be employed, including NAA, NAA+2,4-D or picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 wk, then transferred to media conducive to maturation of embryoids. Cultures are transferred every 2 wk on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soiless plant growth mix, and hardened, e.g., in an environmentally controlled chamber, for example, at about 85% relative humidity, 600 ppm CO₂, and 25-250 microeinsteins m²/s of light. Plants may be matured in a growth chamber or greenhouse. Plants can be regenerated from about 6 wk to 10 months after a transformant is identified, depending on the initial tissue. During regeneration, cells are grown on solid media in tissue culture vessels. Illustrative embodiments of such vessels are petri dishes and Plant Cons. Regenerating plants can be grown at about 19 to 28° C. After the regenerating plants have reached the stage of shoot and root development, they may be transferred to a greenhouse for further growth and testing.

Seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 wk on media containing the above ingredients along with 10-5M abscisic acid and then transferred to growth regulator-free medium for germination.

C. Characterization

To confirm the presence of the exogenous DNA or “transgene(s)” in the regenerating plants, a variety of assays may be performed. Such assays include, for example, “molecular biological” assays, such as Southern and Northern blotting and PCR™; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

D. DNA Integration, RNA Expression and Inheritance

Genomic DNA may be isolated from cell lines or any plant parts to determine the presence of the exogenous gene through the use of techniques well known to those skilled in the art. Note, that intact sequences will not always be present, presumably due to rearrangement or deletion of sequences in the cell. The presence of DNA elements introduced through the methods of this invention may be determined, for example, by polymerase chain reaction (PCR). Using this technique, discreet fragments of DNA are amplified and detected by gel electrophoresis. This type of analysis permits one to determine whether a gene is present in a stable transformant, but does not prove integration of the introduced gene into the host cell genome. It is typically the case, however, that DNA has been integrated into the genome of all transformants that demonstrate the presence of the gene through PCR analysis. In addition, it is not typically possible using PCR techniques to determine whether transformants have exogenous genes introduced into different sites in the genome, i.e., whether transformants are of independent origin. It is contemplated that using PCR techniques it would be possible to clone fragments of the host genomic DNA adjacent to an introduced gene.

Positive proof of DNA integration into the host genome and the independent identities of transformants may be determined using the technique of Southern hybridization. Using this technique, specific DNA sequences introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR, e.g., the presence of a gene, but also demonstrates integration into the genome and characterizes each individual transformant.

It is contemplated that using the techniques of dot or slot blot hybridization which are modifications of Southern hybridization techniques one could obtain the same information that is derived from PCR, e.g., the presence of a gene.

Both PCR and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR techniques also may be used for detection and quantitation of RNA produced from introduced genes. In this application of PCR it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species.

While Southern blotting and PCR may be used to detect the gene(s) in question, they do not provide information as to whether the corresponding protein is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and 14C-acetyl CoA or for anthranilate synthase activity by following loss of fluorescence of anthranilate, to name two.

Very frequently the expression of a gene product is determined by evaluating the phenotypic results of its expression. These assays also may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

### V. Breeding Plants of the Invention

In addition to direct transformation of a particular plant genotype with a construct prepared according to the current invention, transgenic plants may be made by crossing a plant having a selected DNA of the invention to a second plant lacking the construct. For example, a transgenic event can be introduced into a particular plant variety by crossing, without the need for ever directly transforming a plant of that given variety. Therefore, the current invention not only encompasses a plant directly transformed or regenerated from cells which have been transformed in accordance with the current invention, but also the progeny of such plants.

As used herein the term “progeny” denotes the offspring of any generation of a parent plant prepared in accordance with the instant invention, wherein the progeny comprises a selected DNA construct. “Crossing” a plant to provide a plant line having one or more added transgenes relative to a starting plant line, as disclosed herein, is defined as the techniques that result in a transgene of the invention being introduced into a plant line by crossing a starting line with a donor plant line that comprises a transgene of the invention. To achieve this one could, for example, perform the following steps:
(a) plant seeds of the first (starting line) and second (donor plant line that comprises a transgene of the invention) parent plants;
(b) grow the seeds of the first and second parent plants into plants that bear flowers;
(c) pollinate a flower from the first parent plant with pollen from the second parent plant; and
(d) harvest seeds produced on the parent plant bearing the fertilized flower.
   Backcrossing is herein defined as the process including the steps of:
(a) crossing a plant of a first genotype containing a desired gene, DNA sequence or element to a plant of a second genotype lacking the desired gene, DNA sequence or element;
(b) selecting one or more progeny plant containing the desired gene, DNA sequence or element;
(c) crossing the progeny plant to a plant of the second genotype; and
(d) repeating steps (b) and (c) for the purpose of transferring a desired DNA sequence from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

### VII. Definitions

Down-regulation: The reduction in the expression of a DNA or RNA transcript and/or the function or activity of a protein relative to a control or naturally-occurring counterpart.

Expression: The combination of intracellular processes, including transcription and translation undergone by a coding DNA molecule such as a structural gene to produce a polypeptide.

Forage crops: Crops including grasses and legumes used as fodder or silage for livestock production.

Genetic Transformation: A process of introducing a DNA sequence or construct (e.g., a vector or expression cassette) into a cell or protoplast in which that exogenous DNA is incorporated into a chromosome or is capable of autonomous replication.

Heterologous: A sequence which is not normally present in a given host genome in the genetic context in which the sequence is currently found In this respect, the sequence may be native to the host genome, but be rearranged with respect to other genetic sequences within the host sequence. For example, a regulatory sequence may be heterologous in that it is linked to a different coding sequence relative to the native regulatory sequence.

Obtaining: When used in conjunction with a transgenic plant cell or transgenic plant, obtaining means either transforming a non-transgenic plant cell or plant to create the transgenic plant cell or plant, or planting transgenic plant seed to produce the transgenic plant cell or plant. Such a transgenic plant seed may be from an R0 transgenic plant or may be from a progeny of any generation thereof that inherits a given transgenic sequence from a starting transgenic parent plant.

Promoter: A recognition site on a DNA sequence or group of DNA sequences that provides an expression control element for a structural gene and to which RNA polymerase specifically binds and initiates RNA synthesis (transcription) of that gene.

R0 transgenic plant: A plant that has been genetically transformed or has been regenerated from a plant cell or cells that have been genetically transformed.

Regeneration: The process of growing a plant from a plant cell (e.g., plant protoplast, callus or explant).

Selected DNA: A DNA segment which one desires to introduce or has introduced into a plant genome by genetic transformation.

Transformation construct: A chimeric DNA molecule which is designed for introduction into a host genome by genetic transformation. Preferred transformation constructs will comprise all of the genetic elements necessary to direct the expression of one or more exogenous genes. In particular embodiments of the instant invention, it may be desirable to introduce a transformation construct into a host cell in the form of an expression cassette.

Transformed cell: A cell the DNA complement of which has been altered by the introduction of an exogenous DNA molecule into that cell.

Transgene: A segment of DNA which has been incorporated into a host genome or is capable of autonomous replication in a host cell and is capable of causing the expression of one or more coding sequences. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the DNA segment.

Transgenic plant: A plant or progeny plant of any subsequent generation derived therefrom, wherein the DNA of the plant or progeny thereof contains an introduced exogenous DNA segment not naturally present in a non-transgenic plant of the same strain. The transgenic plant may additionally contain sequences which are native to the plant being transformed, but wherein the “exogenous” gene has been altered in order to alter the level or pattern of expression of the gene, for example, by use of one or more heterologous regulatory or other elements.

Vector: A DNA molecule designed for transformation into a host cell. Some vectors may be capable of replication in a host cell. A plasmid is an exemplary vector, as are expression cassettes isolated therefrom.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Identification and Phenotypic Characterization of a M. truncatula Stay-Green Mutant During Natural and Dark-Induced Senescence

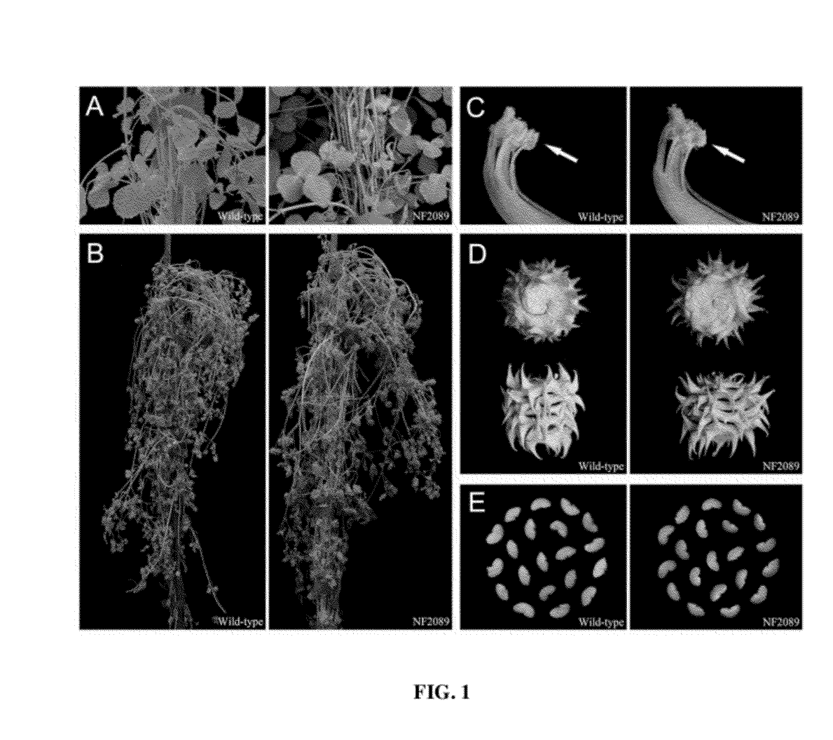

During natural senescence, the basal leaves of wild-type M. truncatula (ecotype R108) turn yellow first, and the whole plant became yellowish gradually. However, the basal leaves of NF2089, an M. truncatula Tnt1 retrotransposon-tagged mutant, were identified as not showing senescent yellow color in the same way as those of wild-type plants (FIG. 1A). Moreover, in this mutant the whole plant remained green until the leaves died (FIG. 1B). Interestingly, the stay-green phenomenon was evident not only in leaves but also in other organs such as anther, central carpels (FIG. 1C), mature pods (FIG. 1D), and seeds (FIG. 1E).

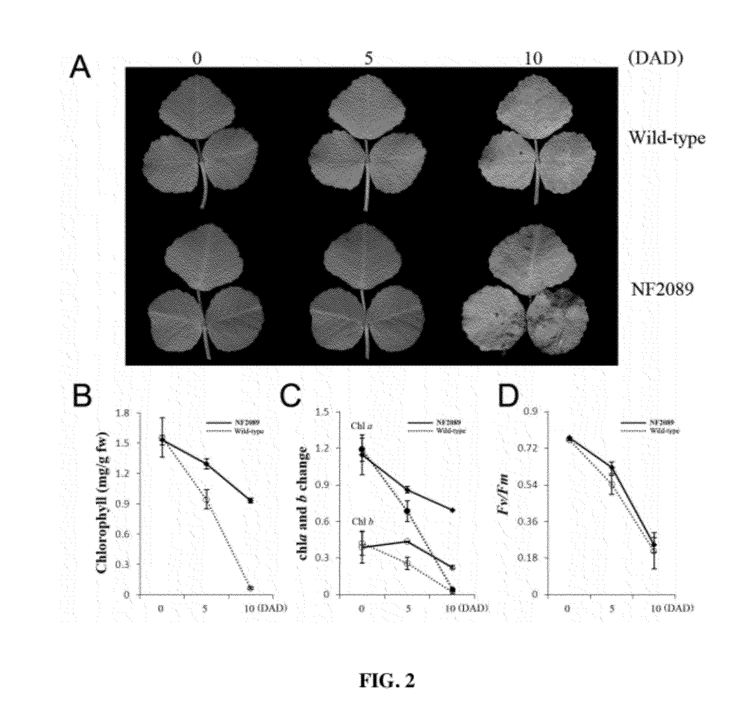

To determine if the NF2089 mutant exhibited a stable stay-green phenotype during dark-induced senescence, detached leaves of the mutant and wild-type were placed in darkness for up to 10 days. The leaves of the mutant remained green on the 5^{th} day after darkness and turned to light green after 10 days of dark treatment. In contrast, wild-type leaves became light green on the 5^{th} day after darkness, and turned yellow after 10 days of dark treatment (FIG. 2A).

To determine chlorophyll content and its possible impacts on leaf photosynthesis in the mutant, whole plants of the mutant and wild-type were transferred to a dark growth chamber. Much of the chlorophyll (Chl) was retained in the mutant after dark treatment (FIG. 2B), with 60% of Chl a and 58% of Chl b retained after 10 days of dark treatment (FIG. 2C). The maximal photochemical efficiency (Fᵥ/Fₘ) of photosystem II (PSII), an important parameter of PSII activity, was also measured (FIG. 2D). The beginning Fᵥ/Fₘ value before dark treatment was similar in both NF2089 and wild-type; while the value of the mutant was slightly higher than that of the wild-type after 5 days of dark treatment, the difference in Fᵥ/Fₘ between mutant and wild-type was not significant after 10 days of darkness. The results indicated that Fᵥ/Fₘ in the mutant decreased in a similar trend as did the wild-type during senescence, although the decrease in the mutant was slightly slower than that of the wild-type.

### Example 2

### Chloroplast Structure and Activity in Senescent Leaves

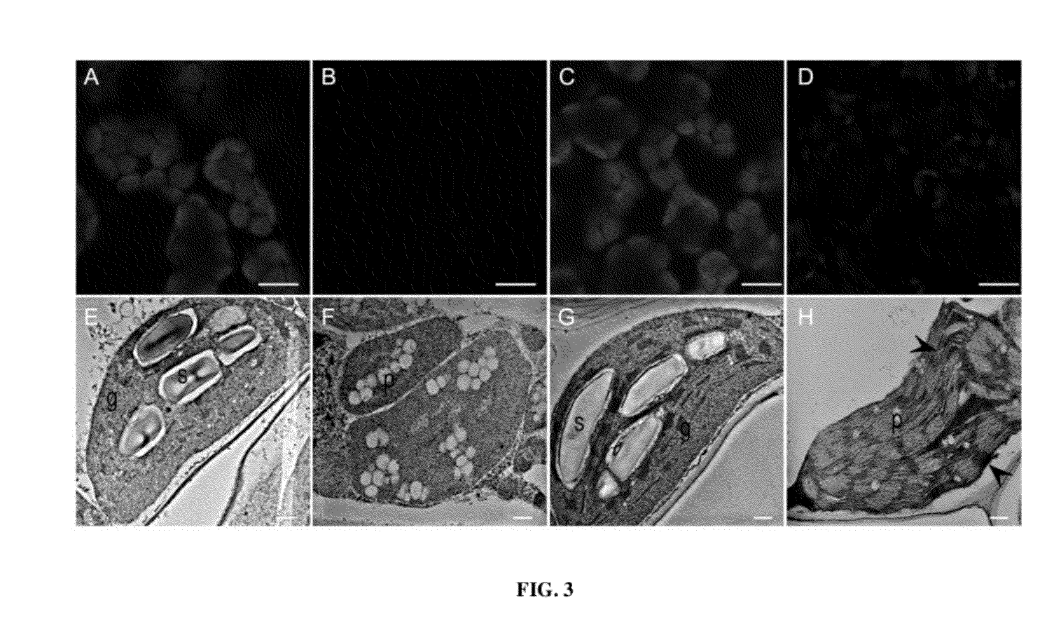

The ultra-structure of chloroplasts and auto-fluorescence of chlorophyll in both wild-type and the NF2089 mutant were examined during dark-induced senescence treatment. Confocal laser scanning microscope (CLSM) was used to observe chlorophyll auto-fluorescence in living and intact mesophyll cells. From CLSM observation, elliptic shapes of chloroplasts with full fluorescence were observed in the wild-type and the mutant (FIG. 3A, C). After 10 days of dark treatment, intensity of auto-fluorescence in wild-type became nearly undetectable (FIG. 3B), indicating severe degradation of chlorophylls. In contrast, auto-fluorescent intensity in NF2089 mutant mesophyll cells remained sufficiently clear that the outlines of some chloroplasts 10 days after dark treatment were observed (FIG. 3D).

Cross-sections of chloroplasts were examined using transmission electron microscopy (TEM), and no obvious difference in chloroplast structure was found between wild-type and NF2089 mutant before senescence treatment (FIGS. 3E and G). After 10 days of dark treatment, shapes of the chloroplasts of both wild-type and NF2089 were round, and plastoglobules became visible. However, the structures of grana stacks were not clear, and thylakoid membranes were disordered in wild-type (FIG. 3F). In contrast, thick and wide grana stacks existed and thylakoid membranes formed strings and layers in chloroplasts of the NF2089 mutant (FIG. 3H). The results indicated that thylakoid membranes in NF2089 were fused with each other to maintain stability during senescence.

### Example 3

### Molecular Cloning and Characterization of SGR Gene in M. truncatula

To determine if the stay-green phenotype in the NF2089 mutant was caused by the mutation of a single gene, the mutant was crossed with wild-type, and segregation analysis was performed in the progeny. The F₁ plants did not show the stay-green phenotype when detached leaves were incubated in darkness. Segregation was observed in F₁ seeds and F₂ plants. The ratio of green versus yellow F₁ seeds was close to 1:3 (148:457), and the ratio between stay-green F₂ plants and non-stay-green F₂ plants was approximately 1:3 (46:143) as shown in Table 1.

TABLE 1
Genetic segregation analysis of the NF2089 mutant

| | Mutant | Wild-type-like | Mutant: |
|---|---|---|---|
| NF2089 × Wild-type | (green color) | (yellow color) | Wild-type-like |
| F₁ seeds | 148 | 457 | 1:3.09 |
| F₁ plants | 46 | 143 | 1:3.11 |
| (detached leaves | | | |
| treated under | | | |
| darkness) | | | |

Furthermore, all the plants from the green seeds showed stay-green phenotype after dark treatment, while the plants from yellow seeds did not show the phenotype. The results demonstrate that NF2089 is a recessive mutant and that the mutation was caused by the loss of function of a single gene. The results also suggest that the stay-green phenotype in the leaves is associated with the green color of the seeds.

To determine which gene was interrupted and associated with the stay-green phenotype in the mutant, Thermal Asymmetric Interlaced Polymerase Chain Reaction (TAIL-PCR) was performed to recover the flanking sequences of the Tnt1 retrotransposon in NF2089. In total, 13 retrotransposon insertions at different sites were recovered from the mutant. One of the insertions (NF2089-9) was found to be associated with the mutation based on PCR (Table 2). BlastN analysis of the flanking sequence against the M. truncatula Gene Index database (MTGI) showed that NF2089-9 was located in the TC126805.

TABLE 2
BlastN analysis of Tnt1 flanking sequences retrieved from the mutant NF2089

| Insertion | Length | | Total | |
|---|---|---|---|---|
| ID | (bp) | Target Description | score | E-Value |
| NF2089-1 | 1001 | M. truncatula clone mth2-11i23 on chromosome | 71.3 | 1e−08 |
| | | 8, complete sequence | | |
| NF2089-2 | 892 | M. truncatula clone mth2-34h6 | 1582 | 0 |
| NF2089-3 | 858 | M. truncatula LYK2 gene, partial sequence | 1162 | 0 |
| NF2089-4 | 824 | M. truncatula clone mth2-38N16 on chromosome | 1097 | 1.00E−91 |
| | | 3, complete sequence | | |
| NF2089-5 | 791 | No hit | — | — |
| NF2089-6 | 741 | M. truncatula chromosome 2 BAC clone mth2- | 80.5 | 1.00E−11 |
| | | 65m5, complete sequence | | |
| NF2089-7 | 668 | M. truncatula chromosome 5 clone mth4-11o22, | 152 | 2.00E−33 |
| | | complete sequence | | |
| NF2089-8 | 678 | G. max (Soybean) symbiotic ammonium | 1644 | 3.00E−91 |
| | | transporter, partial | | |
| NF2089-9 | 520 | P. sativum SGR gene for senescence-inducible | 497 | 3.00E−137 |
| | | chloroplast stay-green protein | | |
| NF2089-10 | 357 | M. truncatula chromosome 5 clone mth2-152j13, | 425 | 5.00E−52 |
| | | complete sequence | | |
| NF2089-11 | 224 | No hit | — | — |
| NF2089-12 | 198 | No hit | — | — |
| NF2089-13 | 93 | M. truncatula chromosome 8 clone mth2-176c9, | 150 | 6.00E−34 |
| | | complete sequence | | |

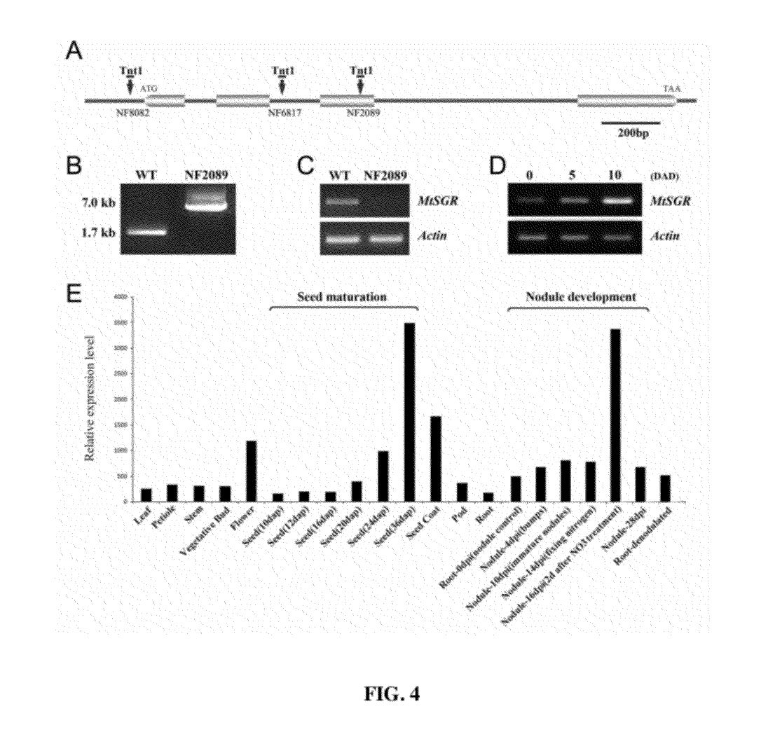

By RT-PCR and sequence analysis, the full-length coding sequence (792 bp) (SEQ ID NO:3) was obtained, which showed 87% identity with PsSGR (Sato et al., 2007), 76% identity with AtNYE1 (Ren et al., 2007), and 65% identity with rice SGR (Park et al., 2007). Sequence analysis of genomic DNA revealed that the gene consists of four exons and three introns (FIG. 4A). PCR amplification of MtSGR in genomic DNA of wild-type (SEQ ID NO:1) and NF2089 confirmed that one 5.3 Kb Tnt1 was inserted into this gene in NF2089 (FIG. 4B), and expression of MtSGR was therefore interrupted in the mutant (FIG. 4C). Analysis of transcription levels of MtSGR in wild-type plants showed that the expression of MtSGR was upregulated during dark-induced senescence (FIG. 4D).

Two other Tnt1 mutants, NF6817 and NF8082, were obtained with the reverse genetics approach. The insertion sites in NF6817 and NF8082 were at the second intron area and the promoter region of MtSGR, respectively (FIG. 4A). Homozygous NF6817 and NF8082 plants exhibited the normal phenotype of the wild-type, suggesting that the Tnt1 insertions of these lines did not interrupt the expression of MtSGR gene.

The expression pattern of MtSGR was analyzed by utilizing the M. truncatula gene expression atlas (Benedito et al., 2008). The expression of MtSGR was detected in all the organs at various developmental stages, with higher levels in mature seed and seed coat and medium to low levels in flowers, petioles, stems, pods, vegetative buds, leaves, roots and young seeds (FIG. 4E). It was interesting to note that the expression level of MtSGR increased during seed maturation. Moreover, the expression level of MtSGR showed a large increase during nodule development and senescence. The highest expression level was observed when seed matured (36 dap) and nodule senesced (2 days after NO₃ treatment) (FIG. 4E). Compared to control, senesced nodules showed a 4-fold increase in the level of MtSGR expression (FIG. 4E).

### Example 4

### Global Expression Profiling of the NF2089 Mutant

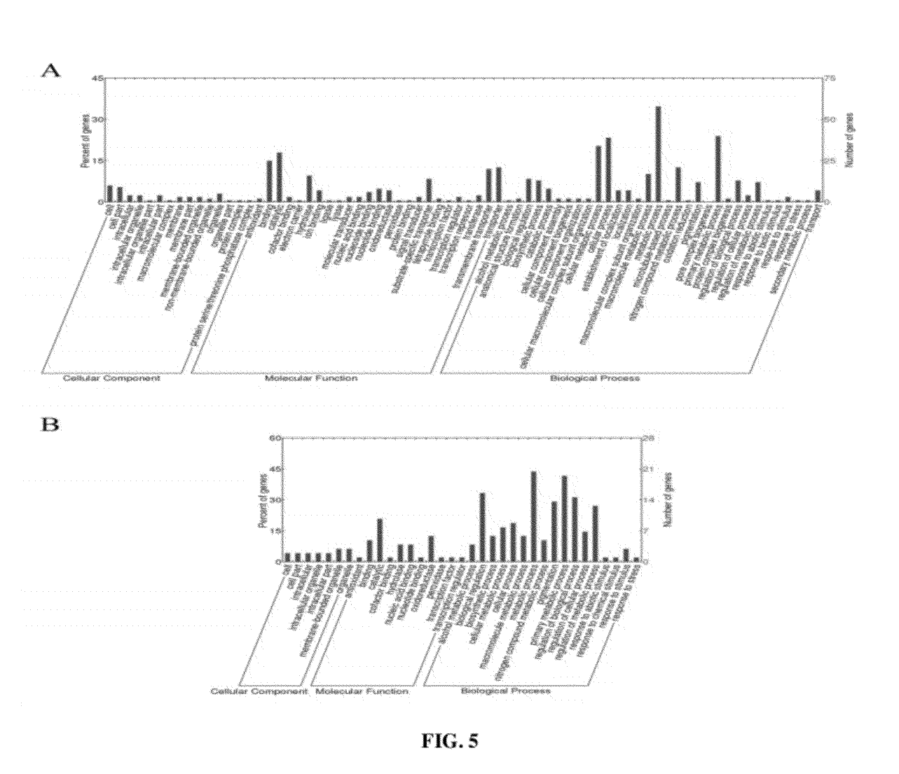

To investigate if MtSGR affected downstream genes in the dark-induced leaf senescence process in M. truncatula, a microarray analysis was performed using Affymetrix Medicago Genome Array. After 5 days of dark treatment, the fully expanded leaves of both the NF2089 mutant and corresponding control were used to isolate RNA for chip analysis. Compared with control plants, 347 genes were repressed, and 1,251 genes were induced by at least 2-fold in the NF2089 mutant. The data suggested that MtSGR mostly plays inhibitory roles on gene expression. The expression level of 12 SENESCENCE-ASSOCIATED GENEs were changed and all of them were up-regulated in NF2089 (Table 3), suggesting that MtSGR functions as a repressor in senescence related pathways in leaves. Some up-regulated genes participate in the process of cellular protein complex assembly, protein polymerization and protein modification by small protein conjugation or removal when the MtSGR is absent (FIG. 5A). However, the expression level of M. truncatula homologs of chlorophyllase (CLH), pheide a oxygenase (PAO), and red Chl catabolite reductase (RCCR) did not show an obvious change, indicating that the absence of MtSGR does not affect the expression of these genes in the chlorophyll degradation pathway.

TABLE 3
Expression level of SENESCENCE ASSOCIATED GENES (SAGs) in the NF2089 mutant

| Probesets | Ratio(NF2089/WT) | Target Description |
|---|---|---|
| Mtr.8529.1.S1_at | 2.070144697 | \| Symbols: SAG101 \| SAG101 (SENESCENCE-ASSOCIATED GENE 101); carboxylesterase/ |
| | | triacylglycerol lipase \| chr5: 4828754-4830769 FORWARD |
| Mtr.37366.1.S1_at | 2.158198117 | \| Symbols: SAG101 \| SAG101 (SENESCENCE-ASSOCIATED GENE 101); carboxylesterase/ |
| | | triacylglycerol lipase \| chr5: 4828754-4830769 FORWARD |
| Mtr.40651.1.S1_at | 2.37634689 | Putative senescence-associated protein [Pisum sativum (Garden pea)] |
| Mtr.5486.1.S1_at | 5.909223103 | \| Symbols: SRG1, ATSRG1 \| SRG1 (SENESCENCE-RELATED GENE 1); oxidoreductase, acting on |
| | | diphenols and related substances as donors, oxygen as acceptor/oxidoreductase, acting on paired donors, |
| | | with incorporation or reduction of molecular oxygen, 2-oxogluta |
| Mtr.37899.1.S1_at | 6.346523138 | \| Symbols: \| senescence-associated protein, putative \| chr3: 17778471-17779299 FORWARD |
| Mtr.10707.1.S1_at | 6.855771411 | \| Symbols: SAG21, AtLEA5 \| SAG21 (SENESCENCE-ASSOCIATED GENE 21) \| chr4: |
| | | 1046414-1046807 REVERSE |
| Mtr.37375.1.S1_at | 7.88547776 | \| Symbols: SRG3 \| SRG3 (senescence-related gene 3); glycerophosphodiester |
| | | phosphodiesterase/phosphoric diester hydrolase \| chr3: 348505-349909 REVERSE |
| Mtr.7463.1.S1_at | 8.859230044 | \| Symbols: SAG21, AtLEA5 \| SAG21 (SENESCENCE-ASSOCIATED GENE 21) \| chr4: |
| | | 1046414-1046807 REVERSE |
| Mtr.11055.1.S1_at | 9.832295963 | \| Symbols: SRG1, ATSRG1 \| SRG1 (SENESCENCE-RELATED GENE 1); oxidoreductase, acting on |
| | | diphenols and related substances as donors, oxygen as acceptor/oxidoreductase, acting on paired donors, |
| | | with incorporation or reduction of molecular oxygen, 2-oxogluta |
| Mtr.5486.1.S1_s_at | 10.03006126 | \| Symbols: SRG1, ATSRG1 \| SRG1 (SENESCENCE-RELATED GENE 1); oxidoreductase, acting on |
| | | diphenols and related substances as donors, oxygen as acceptor/oxidoreductase, acting on paired donors, |
| | | with incorporation or reduction of molecular oxygen, 2-oxogluta |
| Mtr.1760.1.S1_s_at | 11.39823058 | \| Symbols: SAG21, AtLEA5 \| SAG21 (SENESCENCE-ASSOCIATED GENE 21) \| chr4: |
| | | 1046414-1046807 REVERSE |
| Mtr.10435.1.S1_s_at | 35.65153795 | \| Symbols: SRG1, ATSRG1 \| SRG1 (SENESCENCE-RELATED GENE 1); oxidoreductase, acting on |
| | | diphenols and related substances as donors, oxygen as acceptor/oxidoreductase, acting on paired donors, |
| | | with incorporation or reduction of molecular oxygen, 2-oxogluta |

TABLE 4
Up- and down-regulated genes in the NF2089 mutant microarray encoding chloroplast-related proteins

| Probesets | Ratio(NF2089/WT) | Target Description |
|---|---|---|
| Mtr.11436.1.S1_at | 0.141079102 | TC109939/FEA = mRNA/DEF = similar to UP\|HIS2_ARATH (O82768) Histidine biosynthesis |
| | | bifunctional protein hisIE, chloroplast precursor [Includes: Phosphoribosyl-AMP cyclohydrolase |
| | | (PRA-CH); Phosphoribosyl-ATP pyrophosphatase (PRA-PH)], partial (72%) |
| Mtr.19818.1.S1_at | 0.148372077 | Ferritin-1, chloroplast precursor [Pisum sativum (Garden pea)] |
| Mtr.45179.1.S1_at | 0.402671786 | TC98992/FEA = mRNA/DEF = homologue to UP\|MDHP_MEDSA (O48902) Malate dehydrogenase |
| | | [NADP], chloroplast precursor (NADP-MDH), partial (26%) |
| Mtr.19267.1.S1_at | 0.405486884 | Photosystem I reaction center subunit II, chloroplast precursor |
| | | [Cucumis sativus (Cucumber)] |
| Mtr.31163.1.S1_at | 0.412690056 | AJ497560/FEA = mRNA/DEF = weakly similar to UP\|ZIP4_ARATH (O04089) Zinc |
| | | transporter 4, chloroplast precursor (ZRT/IRT-like protein 4), partial (14%) |
| Mtr.13254.1.S1_at | 0.424094253 | \| Symbols: CIA2 \| CIA2 (CHLOROPLAST IMPORT APPARATUS 2); transcription regulator \| |
| | | chr5: 23168393-23169523 FORWARD |
| Mtr.39235.1.S1_at | 0.438010252 | \| Symbols: \| FUNCTIONS IN: molecular_function unknown; INVOLVED IN: biological_process |
| | | unknown; LOCATED IN: chloroplast; EXPRESSED IN: 21 plant structures; EXPRESSED DURING: |
| | | 13 growth stages; BEST Arabidopsis thaliana protein match is: |
| | | LPA1 (LOW PSII ACCU |
| Mtr.41668.1.S1_at | 0.461032362 | Ribulose bisphosphate carboxylase/oxygenase activase 1, chloroplast, putative n = 1 |
| | | Tax = Ricinus communis RepID = B9T427_RICCO |
| Mtr.40149.1.S1_at | 0.465154865 | TC106620/FEA = mRNA/DEF = similar to UP\|THI4_CITSI (O23787) Thiazole |
| | | biosynthetic enzyme, chloroplast precursor, partial (85%) |
| Msa.2574.1.S1_at | 0.471134824 | similar to UniRef100_O23787 Cluster: Thiazole biosynthetic enzyme, chloroplast precursor; |
| | | n = 1; Citrus sinensis\|Rep: Thiazole biosynthetic enzyme, chloroplast |
| | | precursor—Citrus sinensis (Sweet orange), partial (85%) |
| Mtr.10569.1.S1_at | 0.472498133 | Photosystem II 5 kDa protein, chloroplast precursor [Gossypium hirsutum (Upland cotton)] |
| Mtr.13203.1.S1_at | 0.475636354 | TC97263/FEA = mRNA/DEF = similar to UP\|TL30_ARATH (O49292) Thylakoid lumenal |
| | | 29.8 kDa protein, chloroplast precursor, partial (62%) |
| Msa.1131.1.S1_at | 0.475926124 | similar to UniRef100_Q41709 Cluster: Ferritin-2, chloroplast precursor; n = 1; |
| | | Vigna unguiculata\|Rep: Ferritin-2, chloroplast |
| | | precursor—Vigna unguiculata (Cowpea), partial (74%) |
| Mtr.21631.1.S1_at | 0.476163055 | \| Symbols: HCF109, ATPRFB \| HCF109 (HIGH CHLOROPHYLL FLUORESCENT 109); translation |
| | | release factor/translation release factor, codon specific \| chr5: 14236083-14237974 |
| | | REVERSE |
| Mtr.10936.1.S1_at | 0.484582923 | \| Symbols: \| FUNCTIONS IN: molecular_function unknown; INVOLVED IN: protein folding, |
| | | protein transport; LOCATED IN: chloroplast; EXPRESSED IN: 23 plant structures; |
| | | EXPRESSED DURING: 13 growth stages; CONTAINS InterPro DOMAIN/s: Trigger factor, ribosome-bi |
| Mtr.12842.1.S1_at | 0.486259328 | \| Symbols: \| FUNCTIONS IN: molecular_function unknown; INVOLVED IN: biological_process |
| | | unknown; LOCATED IN: thylakoid, chloroplast thylakoid membrane, chloroplast; EXPRESSED IN: |
| | | 21 plant structures; EXPRESSED DURING: 13 growth stages; CONTAINS InterPro DO |
| Mtr.34428.1.S1_s_at | 0.490767461 | \| Symbols: GAPB \| GAPB (GLYCERALDEHYDE-3-PHOSPHATE DEHYDROGENASE B SUBUNIT); |
| | | glyceraldehyde-3-phosphate dehydrogenase (NADP+)/glyceraldehyde-3-phosphate |
| | | dehydrogenase \| chr1: 16127552-16129584 FORWARD |
| Mtr.38712.1.S1_at | 0.491451464 | \| Symbols: \| FUNCTIONS IN: molecular_function unknown; INVOLVED IN: |
| | | biological_process unknown; LOCATED IN: chloroplast; EXPRESSED IN: 21 plant structures; |
| | | EXPRESSED DURING: 13 growth stages; BEST Arabidopsis thaliana protein match is: |
| | | LPA1 (LOW PSII ACCU |
| Mtr.40230.1.S1_at | 0.491909739 | TC106811/FEA = mRNA/DEF = homologue to UP\|PSAG_ARATH (Q9S7N7) Photosystem I |
| | | reaction center subunit V, chloroplast precursor (PSI-G), partial (67%) |
| Mtr.37448.1.S1_at | 0.49249625 | Photosystem II 5 kDa protein, chloroplast precursor [Gossypium hirsutum |
| | | (Upland cotton)] |
| Msa.981.1.S1_at | 0.492955422 | similar to UniRef100_O23787 Cluster: Thiazole biosynthetic enzyme, chloroplast precursor; |
| | | n = 1; Citrus sinensis\|Rep: Thiazole biosynthetic enzyme, |
| | | chloroplast precursor—Citrus sinensis (Sweet orange), partial (85%) |
| Mtr.12355.1.S1_at | 0.49823744 | TC94500/FEA = mRNA/DEF = similar to UP\|FRI2_VIGUN (Q41709) Ferritin 2, |
| | | chloroplast precursor, partial (84%) |
| Mtr.42994.1.S1_at | 2.107117193 | TC94286/FEA = mRNA/DEF = homologue to UP\|ATPG_PEA (P28552) ATP synthase gamma |
| | | chain, chloroplast precursor, partial (98%) |
| Mtr.33759.1.S1_at | 2.358557815 | \| Symbols: \| LOCATED IN: chloroplast; EXPRESSED IN: flower, pollen tube; |
| | | EXPRESSED DURING: petal differentiation and expansion stage; BEST |
| | | Arabidopsis thaliana protein match is: F-box family protein |
| | | (TAIR: AT4G22030.1); Has 51 Blast hits to 51 proteins in |
| Mtr.37165.1.S1_at | 2.363878307 | \| Symbols: \| FUNCTIONS IN: molecular_function unknown; INVOLVED IN: |
| | | biological_process unknown; LOCATED IN: chloroplast; CONTAINS InterPro DOMAIN/s: |
| | | Cyclin-like F-box (InterPro: IPR001810); BEST Arabidopsis thaliana protein |
| | | match is: F-box family protein ( |
| Msa.995.1.S1_at | 2.473846563 | Chloroplast cystathionine beta lyase (Fragment) n = 1 |
| | | Tax = Medicago sativa RepID = Q6J9X7_MEDSA |
| Mtr.43119.1.S1_at | 2.53908513 | TC94577/FEA = mRNA/DEF = similar to UP\|ARC1_LYCES (Q42884) Chorismate |
| | | synthase 1, chloroplast precursor (5-enolpyruvylshikimate-3-phosphate phospholyase 1), |
| | | partial (87%) |
| Mtr.38042.1.S1_at | 2.605234321 | TC101953/FEA = mRNA/DEF = similar to UP\|METC_ARATH (P53780) Cystathionine |
| | | beta-lyase, chloroplast precursor (CBL) (Beta-cystathionase) (Cysteine lyase), partial (82%) |
| Mtr.11140.1.S1_at | 2.662478682 | TC109022/FEA = mRNA/DEF = similar to UP\|APKB_ARATH (P46573) Protein kinase |
| | | APK1B, chloroplast precursor, partial (50%) |
| Mtr.9107.1.S1_at | 2.739530049 | \| Symbols: \| FUNCTIONS IN: molecular_function unknown; INVOLVED IN: |
| | | biological_process unknown; LOCATED IN: chloroplast; CONTAINS InterPro DOMAIN/s: |
| | | Cyclin-like F-box (InterPro: IPR001810); BEST Arabidopsis thaliana protein |
| | | match is: F-box family protein ( |
| Mtr.51996.1.S1_at | 3.193516589 | homologue to UniRef100_O65194 Cluster: Ribulose bisphosphate carboxylase small chain, |
| | | chloroplast precursor; n = 1; Medicago sativa\|Rep: Ribulose |
| | | bisphosphate carboxylase small chain, chloroplast precursor—Medicago sativa |
| | | (Alfalfa), partial (98%) |
| Mtr.9938.1.S1_at | 3.354560369 | TC104914/FEA = mRNA/DEF = similar to UP\|DAPA_SOYBN (Q42800) |
| | | Dihydrodipicolinate synthase, chloroplast precursor (DHDPS), partial (36%) |
| Mtr.25311.1.S1_at | 4.155022016 | 1804.m00040/FEA = mRNA/DEF = CR932965.1 85362 91247 mth2-47e6 weakly similar to |
| | | TIGR_Ath1\|At2g17760-GOpep .1 68409.m01853 chloroplast nucleoid DNA-binding |
| | | protein-related contains, partial (97%) |
| Mtr.45405.1.S1_at | 5.505654686 | homologue to UniRef100_A4UTS2 Cluster: Chloroplast glucose-6-phosphate/phosphate translocator; |
| | | n = 1; Pisum sativum\|Rep: Chloroplast |
| | | glucose-6-phosphate/phosphate translocator—Pisum sativum (Garden pea), |
| | | partial (81%) |
| Mtr.24739.1.S1_at | 5.78508013 | Phosphoserine aminotransferase, chloroplast precursor [Arabidopsis thaliana |
| | | (Mouse-ear cress)] |
| Mtr.11516.1.S1_at | 5.926006536 | Phosphoserine aminotransferase, chloroplast precursor [Spinacia oleracea |
| | | (Spinach)] |
| Mtr.12916.1.S1_at | 6.377107135 | \| Symbols: \| FUNCTIONS IN: molecular_function unknown; INVOLVED IN: |
| | | biological_process unknown; LOCATED IN: chloroplast; EXPRESSED IN: 23 plant structures; |
| | | EXPRESSED DURING: 13 growth stages; CONTAINS InterPro DOMAIN/s: Uncharacterised conserved |
| | | protein U |
| Mtr.8645.1.S1_at | 9.39199443 | TC100898/FEA = mRNA/DEF = similar to UP\|GCH2_ARATH (P47924) Riboflavin |
| | | biosynthesis protein ribA, chloroplast precursor [Includes: GTP cyclohydrolase II; |
| | | 3,4-dihydroxy-2-butanone 4-phosphate synthase (DHBP synthase)], partial (75%) |
| Mtr.10536.1.S1_at | 11.32425778 | similar to UniRef100_A7XZV0 Cluster: Chloroplast omega-3 fatty acid desaturase; n = 1; |
| | | Jatropha curcas\|Rep: Chloroplast omega-3 fatty acid |
| | | desaturase—Jatropha curcas, partial (73%) |
| Mtr.10416.1.S1_at | 21.83576754 | homologue to UniRef100_P27608 Cluster: Phospho-2-dehydro-3-deoxyheptonate aldolase 1, |
| | | chloroplast precursor; n = 1; Nicotiana tabacum\|Rep: |
| | | Phospho-2-dehydro-3-deoxyheptonate aldolase 1, chloroplast |
| | | precursor—Nicotiana tabacum (Common tobacco), partial (83 |

Only 2.5% of the up- or down-regulated genes (40/1,598) belong to chloroplast-related proteins (Table 4). The result implies that MtSGR may participate in broad biological processes during leaf senescence besides chlorophyll degradation. To further understand the biological events of MtSGR involved in the dark-induced leaf senescence process in M. truncatula, Gene Ontology (GO) analysis were performed using Gene Ontology Enrichment Analysis Software Toolkit (“GO-EAST”; Zheng and Wang, 2008). The results showed that 168 GO classes and 49 GO classes were enriched, respectively, in up-regulated and down-regulated genes in the NF2089 mutant. Many biological processes, cellular components, and molecular functions were found to be substantially affected in senescing leaves when MtSGR was absent. The involvement of multiple function categories suggests that MtSGR plays a broad role in plant development and senescence.

The representations of probe sets in the enriched GO classes were examined with Web Gene Ontology Annotation Plotting (“WEGO”; Ye et al., 2006) and the percentage of probe sets in each of the GO terms was calculated. In the GO main category titled Biological Process, which includes the subcategories of “metabolic process,” “cellular process,” “primary metabolic process,” “cellular metabolic process,” “nitrogen compound metabolic process,” and “biosynthetic process,” approximately 34.7% of the up-regulated probe sets were grouped into the “metabolic process” subcategory (FIG. 5A). The “cellular process” subcategory, which involves cell growth and/or maintenance, is the second highest category (23.4% of up-regulated probe sets) in Biological Process. The category has subcategories that are involved in cellular metabolic process, cellular macromolecular complex subunit organization and microtubule-based process, etc. (FIG. 5A). The “metabolic process” and “cellular process” categories have some overlapping subcategories, such as those belonging to the cellular metabolic process. In addition, the data also showed that the up-regulated genes are involved in the category of pigmentation (7.2% of examined probe sets) and response to stimulus (1.8% of examined probe sets). It is interesting to note that the down-regulated genes participated in similar subcategories of the main category Biological Process, including metabolic process (43.8%), cellular process (18.8%), pigmentation (29.2%), and response to stimulus (6.3%), in which up-regulated genes are also involved (FIG. 5B). These data imply that MtSGR affects gene expression in some specified processes. Taken together, the findings of the global expression profiling and GO analysis suggest that MtSGR affects gene expression in a genome-wide scale and a function-dependent manner.

### Example 5

### Isolation of SGR Gene from Alfalfa (M. sativa)

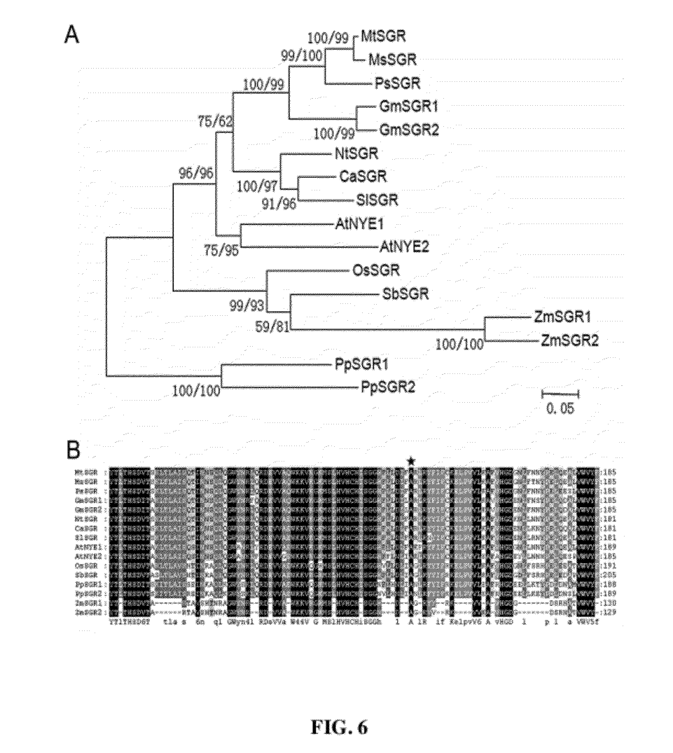

The SGR gene (SEQ ID NO:3) was cloned from alfalfa and designated as MsSGR based on high sequence similarity between M. truncatula and M. sativa. Analysis of deduced amino acid sequences revealed that MsSGR encodes a protein of 263 amino acids, with predicted isoelectric point of 8.716 and molecular weight of 30 kDa. Predicted SGR protein sequences from several species were collected from GenBank and used for phylogenetic analysis (FIG. 6). The phylogenetic trees were rooted by the P. patens (moss) SGR and divided into two clades: one belonging to monocotyledon species and the other belonging to dicotyledon species. Phylogenetic analysis showed that MsSGR and MtSGR were closest to each other and were clustered closely to PsSGR (SEQ ID NO:27) (see FIG. 6A). The MsSGR sequence showed 98% identity to MtSGR, 86% to PsSGR, 78% to GmSGR1 (SEQ ID NO:23), 67% to AtNYE1 (SEQ ID NO:19), and 64% to rice SGR (SEQ ID NO:33). The SGR family members share a highly conserved central region but are divergent at N and C termini. The amino acid Arg-145 (FIG. 6B, denoted by an asterisk), the site at which the Tnt1 was inserted in the mutant NF2089, is an invariant residue within the SGR family.

### Example 6

### Expression Level of MsSGR in Senesced Nodules of Alfalfa

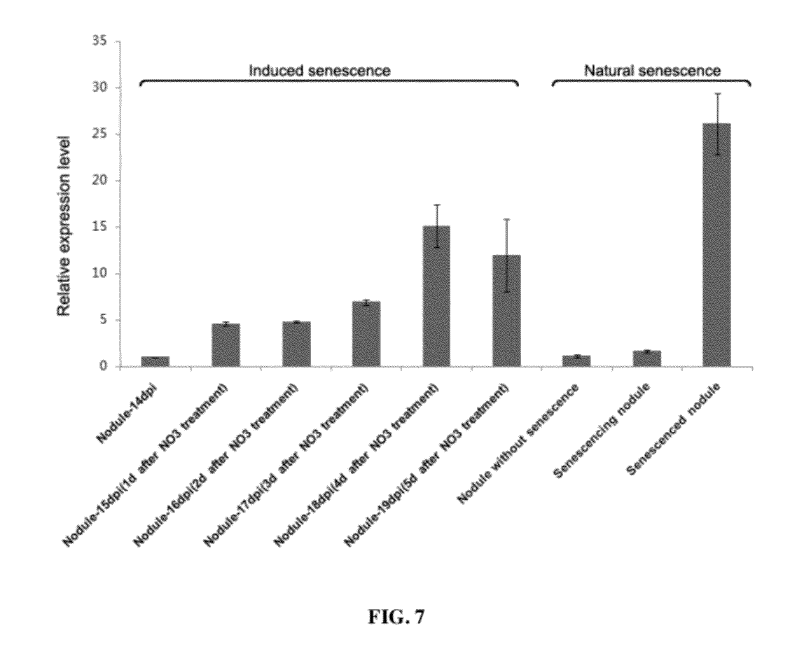

To confirm the relationship between SGR gene expression and nodule senescence, quantitative RT-PCR was used to analyze the expression level of MsSGR in NO₃-induced nodule senescence and natural nodule senescence in alfalfa. Compared with expression level in 14 dpi nodules, the accumulation of MsSGR transcripts increased gradually in the senesced nodules after NO₃ treatment and showed a 15-fold increase at the 4^{th} day and a 12-fold increase at the 5^{th} day (FIG. 7). Similar results were obtained in naturally senesced nodules. The expression of MsSGR was slightly induced in the nodules showing a senescing zone as the nodules age (senescing nodule) (Van de Velde et al., 2006) and then dramatically increased 23-fold in fully senesced nodules compared to non-senesced nodules (FIG. 7).

### Example 7

### Down-Regulation of MsSGR in Alfalfa by RNA Interference

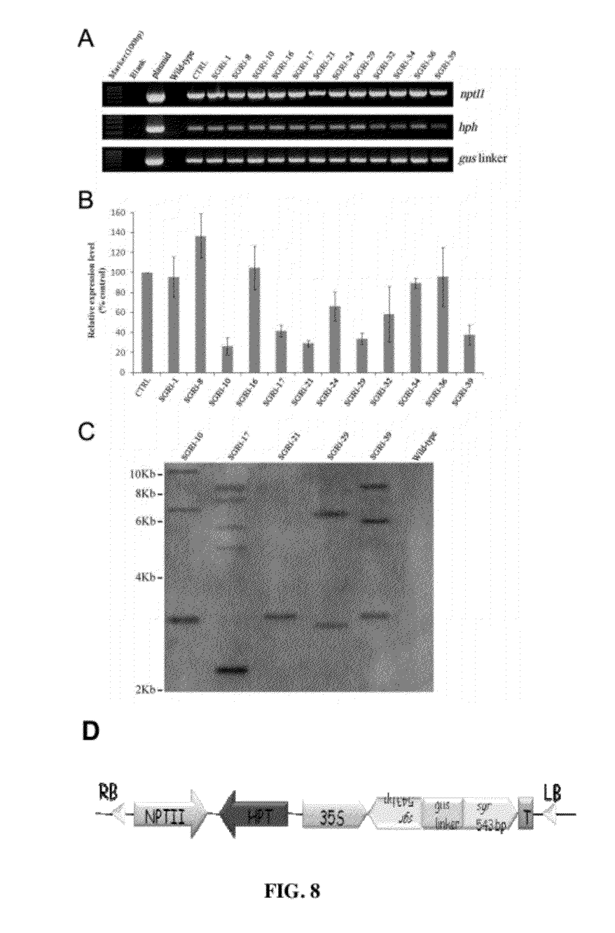

To repress the function of endogenous MsSGR gene, a MsSGR-RNAi vector was constructed and introduced into alfalfa by Agrobacterium-mediated transformation. To construct the RNAi vector, a 543 bp fragment of SGR (SEQ ID NO:5) was PCR-amplified from alfalfa using primers MsSGR-F and MsSGR-R. The fragment was inserted into pENTR™/D-TOPO® cloning vector and transferred into the pANDA35HK vector by LR recombination reactions (Invitrogen, Chicago, Ill.). The final binary vector shown schematically in FIG. 8D was transferred into A. tumefaciens strain EHA105 using freezing/heat shock method.

Twenty transgenic lines were identified through PCR analysis (FIG. 8A). Quantitative real-time PCR analysis revealed that five transgenic lines, SGRi-10, SGRi-17, SGRi-21, SGRi-29, and SGRi-39, had their SGR transcript effectively down-regulated by more than 60% when compared to the empty vector control line (CTRL) (FIG. 8B). These five transgenic alfalfa lines were used for further analyses.

Southern blot hybridization analysis confirmed that the transgene was stably integrated in the alfalfa genome and that the regenerated positive lines were truly independent transformants. Both single copy and multiple copy integrations of the transgene were observed in the transgenic lines. Transgenic line SGRi-21 had single copy integration, SGRi-29 contained two copies of the transgene, and SGRi-10, SGRi-17, and SGRi-39 had at least three copies of the transgene (FIG. 8C).

A. Senescence of the MsSGR-RNAi Transgenic Alfalfa Plants

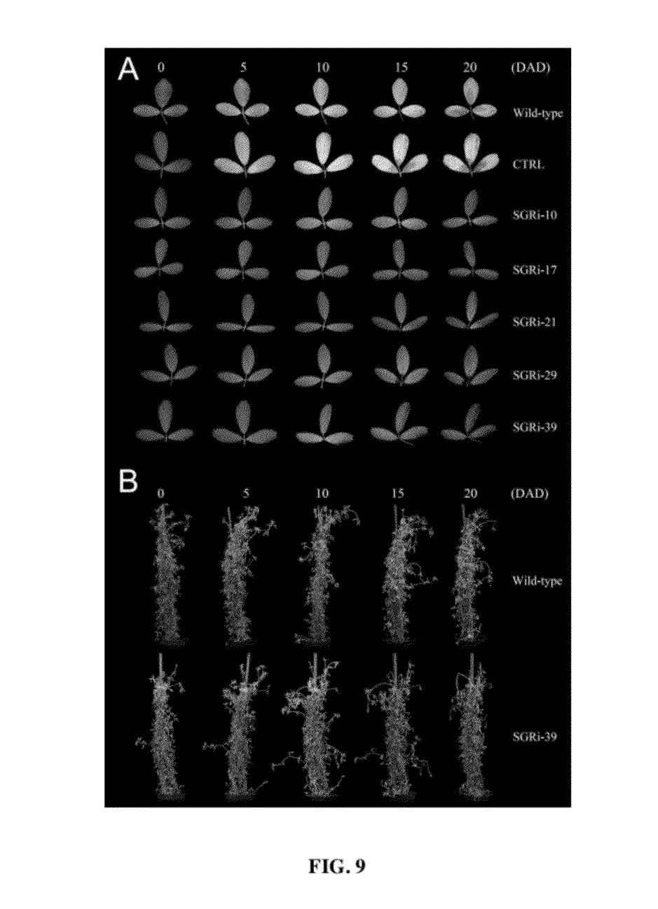

The effects of MsSGR down-regulation on transgenic alfalfa were analyzed by incubating detached leaves in darkness to induce senescence. The transgenic lines exhibited a stable non-yellowing phenotype during a continuous 20-day dark treatment. Although the detached leaves from the transgenics turned light green on the 5^{th} day of dark incubation, the green color remained until the end of the whole treatment process, as shown in FIG. 9A. In contrast, the empty vector control and wild-type leaves began to turn yellow as early as on the 5^{th} day, and the yellow color spread gradually to full leaves by the 10^{th} day of dark treatment.

To determine the effects of MsSGR suppression at the whole plant level, live transgenic plants were placed in a dark growth chamber. During natural senescence of alfalfa plants, the lower leaves usually senesce earlier than do the upper leaves. A similar phenomenon was observed in dark-induced senescence in alfalfa. On the 5^{th} day of dark treatment, basal leaves of the control plants began to turn yellow, while the leaves of all the transgenic lines remained green. On the 10^{th} day of dark treatment, whole plants of the controls became yellowish, with some basal leaves turning red, while the transgenic lines kept the stay-green phenotype although some of the leaves at the top became wrinkled. After 15 and 20 days of dark treatment, almost all the leaves in the control became yellow and red while the leaves of the transgenic lines remained similar green color to that of the 10^{th} day of dark treatment (FIG. 9B).

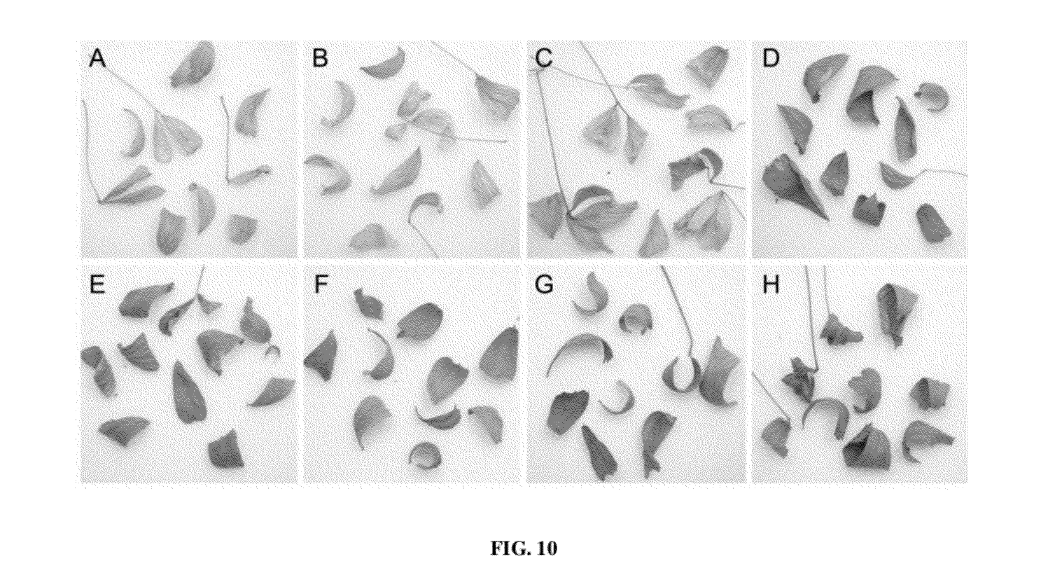

Under natural growth conditions, as a plant grew, the old leaves lost function and dropped off from the plant. A color difference was observed in these fallen leaves, with control leaves showing yellow color while transgenic leaves remained green (FIG. 10).

B. Chloroplast Structure and Activity in Alfalfa Transgenic Lines

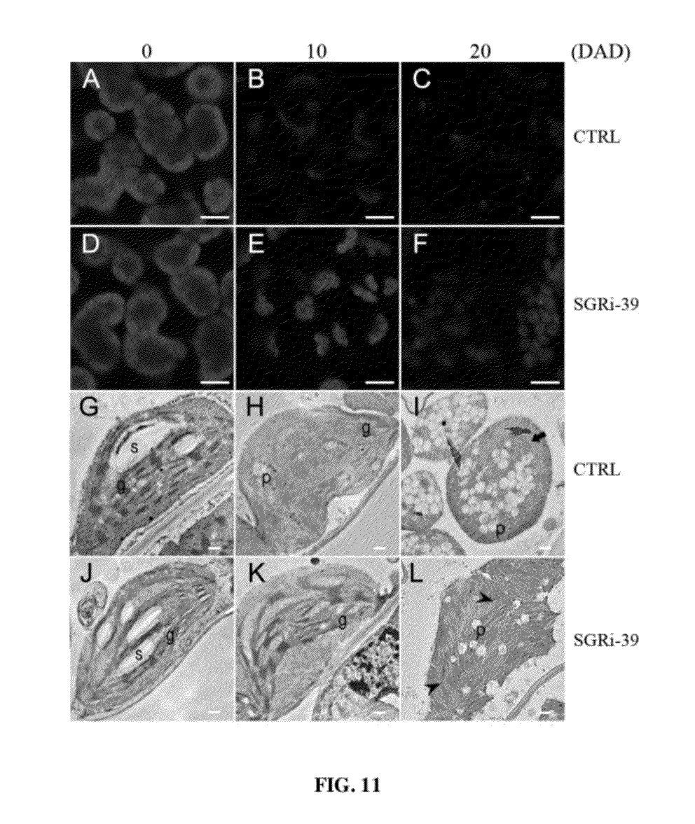

CLSM observation showed that the whole senescent progression of the auto-fluorescence intensity in the alfalfa transgenic lines was similar to that observed in NF2089 (FIG. 11A-F). TEM analysis (FIG. 11G-L) revealed that chloroplast outline, shapes, and content as well as thickness of grana stacks in the transgenic alfalfa lines showed similar temporal changes as did M. truncatula NF2089 during dark-induced senescence.

C. Chlorophyll Content and Physiological Changes in Transgenic Alfalfa During Dark Incubation

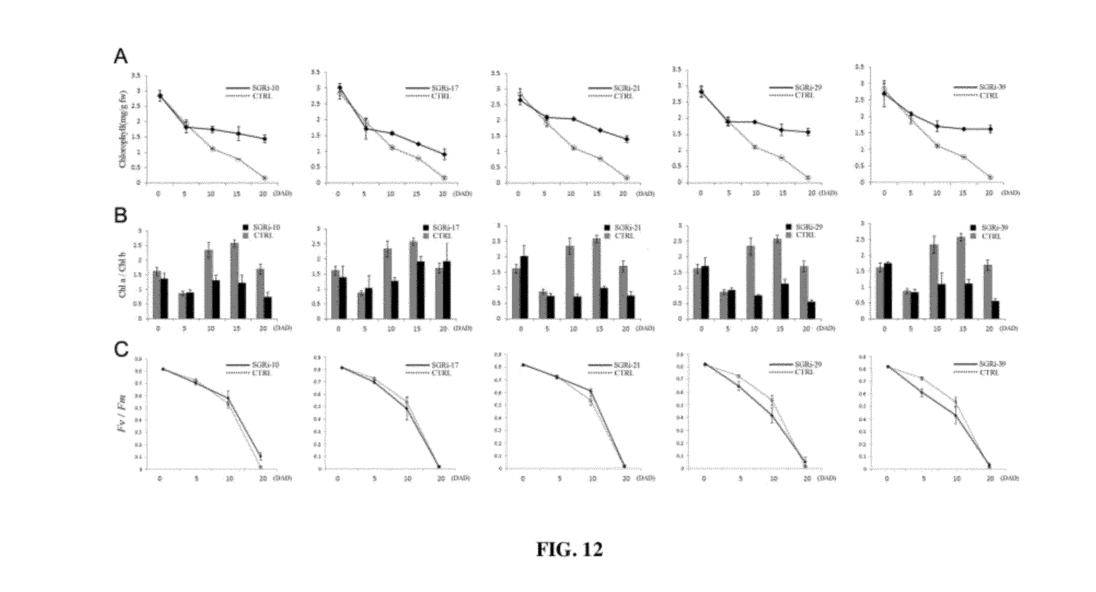

Chlorophyll content, chl a/b ratio, and PSII functionality (measured as Fᵥ/Fₘ) were measured during dark treatment of living transgenic alfalfa plants. No difference was observed in chlorophyll loss after 5 days of dark treatment (FIG. 12A). Significant changes were observed, however, after 10 days of dark treatment. By the 20^{th} day of dark treatment, only 5% of chlorophylls were left in the control leaves while more than 50% of chlorophylls were maintained in most of the transgenics (50.6% in SGRi 10, 52.8% in SGRi 21, 55.6% in SGRi 29, 60.2% in SGRi 39). Among the transgenic lines, SGRi 39 retained the highest level of chlorophylls (60.2%), which was double the amount of chlorophylls retained in SGRi 17 (30.0%).

Chl a/b ratio of each transgenic line reflects the relative abundance of the two types of chlorophylls during senescence. No difference was observed between transgenic and control plants after 0 and 5 days of dark treatment. However, after 10 to 20 days of darkness, the transgenics showed large decreases in chl a/b ratios (FIG. 12B). As shown in FIG. 12C, Fᵥ/Fₘ values decreased similarly during the course of senescence in the transgenic lines and the control.

D. Improvement of Forage Quality of MsSGR-RNAi Transgenic Alfalfa

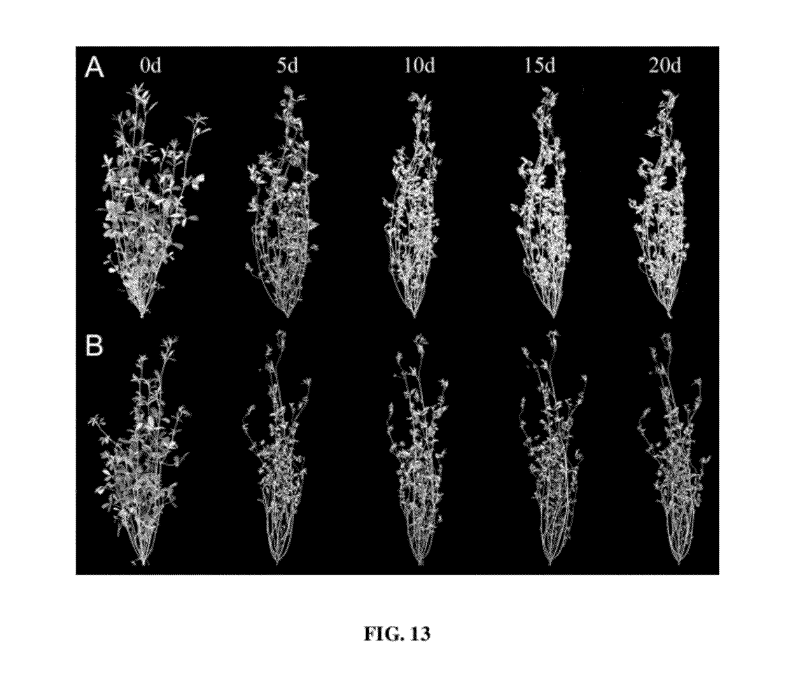

The process for alfalfa hay harvesting is usually fresh-cut, dried and baled in field. To evaluate the potential application of MsSGR-RNAi alfalfa, the harvest and drying process was mimicked using these transgenic materials. The transgenics were greener and fresher than the control after 5 days of drying. The difference in visual quality of the transgenics and the control became more obvious after 10 days of drying, with transgenics showing a more attractive greenish color (FIG. 13).

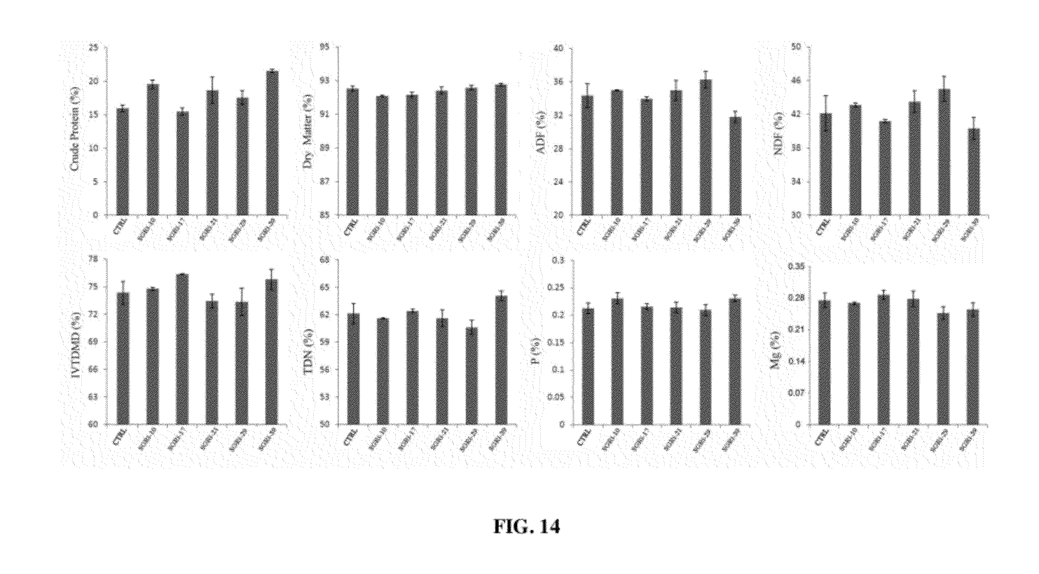

Forage nutritive quality analysis revealed that most of the transgenic lines (except SGRi-17) had increased crude protein content (FIG. 14). Compared to the control, the level of increase in crude protein content of SGRi-29, -21, -10 and -39 varied from 1.6% to 5.6%. Other nutritive quality traits, including in vitro true dry matter digestibility (IVTDMD), acid detergent fiber (ADF) content, neutral detergent fiber (NDF) content, total digestible nutrients (TDN), phosphorus (P) concentration, and magnesium (Mg) concentration did not show significant changes between the transgenics and the control.

### Example 8

### Materials and Methods

A. Plant Material and Growth Conditions

Generation of the M. truncatula Tnt1 insertional mutant population was described previously by Tadege et al. (2008). M. truncatula ecotype R108 was used as wild-type. Mutant and wild-type seeds were scarified with concentrated sulfuric acid and treated at 4° C. for 5 days on filter paper. Small plantlets were transferred to Metro-Mix 830 soil mix and grown in the greenhouse or growth chamber under the following conditions: 24° C. day/22° C. night temperature; 16-h/8-h photoperiod; 70% relative humidity, and 150 μmol/m²/s light intensity.

An alfalfa (M. sativa) genotype Regen SY-4D (RSY-4D) was used for Agrobacterium-mediated transformation to produce transgenic plants. Both transgenic and wild-type alfalfa plants were vegetatively propagated using shoot cuttings. All plants were grown in a greenhouse at 24° C./22° C. with 16-h/8-h photoperiod and relative humidity of 70%.

Both detached leaves and whole plants were used for dark-induced senescence experiments. For senescence treatment of detached leaves, fully expanded leaves were excised from 4- to 5-week old M. truncatula and alfalfa plants and then incubated in sterilized wet filter paper at 25° C. in the dark for up to 20 days. For senescence treatment of living plants, 5-week old plants were kept in a growth chamber without light.

To mimic the harvesting and drying process of alfalfa hay, aboveground parts of transgenic and control alfalfa lines were cut and placed outside under well-ventilated conditions and the day temperature averaged about 35° C.

B. Screening of M. truncatula Stay-Green Mutant and Cloning of MtSGR

The NF2089 mutant line was identified from a Tnt1 insertional population (more than 10,000 lines) of M. truncatula based on the segregation of leaf and seed color, green versus yellow. Tnt1 flanking sequences of mutant NF2089 were recovered using the method of Thermal Asymmetric Interlaced Polymerase Chain Reaction (TAIL-PCR) (Liu et al., 2005). The PCR products were purified and cloned into pGEM-T Easy vector (Promega) for sequencing. The flanking sequences were blasted against the M. truncatula genome sequence at the National Center for Biotechnology Information (Table 2). Based on the flanking sequences recovered at the 9^{th} insertion, the electronic sequence of MtSGR was obtained from M. truncatula Gene Index (MTGI; compbio.dfci.harvard.edu/tgi/cgi-bin/tgi/gimain.pl?gudb=medicago). Genomic sequences of MtSGR were obtained through PCR amplification using primers Mtsgr-F and Mtsgr-R (Table 5).

The coding sequences of MtSGR were PCR amplified with primers MtSGR-F and MtSGR-R. The amplifications were performed using Ex Taq polymerase (TaKaRa Ex Taq™, Code No. RR001A, Osaka, Japan). The Tnt1 insertion site in the M. truncatula genome was confirmed using primers Mtsgr-F, Mtsgr-R coupled with Tnt1 border primers Tnt1-F and Tnt1-R (Table 5; the “F” and “R” designations refers to “forward” and “reverse”, respectively) Homozygous or heterozygous status of MtSGR in M. truncatula plants was checked using the primer pair Mtsgr-F and Mtsgr-R. Primers Mtsgr-2F and -2R were coupled with Tnt1 border primers for reverse screening for other sgr mutants. Two other Tnt1 mutants, NF8082 and NF6817, were identified.

TABLE 5
Primer sequences.

| Name | Primer sequences |
|---|---|
| Mtsgr-F | TCCGCCACCGTTGGATTAGAG (SEQ ID NO: 6) |
| Mtsgr-R | TTGGATTTTCCCACGATGAGC (SEQ ID NO: 7) |
| Mtsgr-2F | ACTCTAACCACCGCTCCT (SEQ ID NO: 8) |
| Mtsgr-2R | TACCCAAACCAATGCTTCCT (SEQ ID NO: 9) |
| MtSGR-F | ATGGGTACTCTAACCACCGCTC (SEQ ID NO: 10) |
| MtSGR-R | TTACAGTGATTGTTGAGTTTCAATTCC (SEQ ID NO: |
| | 11) |
| MsSGR-F | CACCACTCTAACCACCGCTCCT (SEQ ID NO: 12) |
| MsSGR-R | TACCCAAACCAATGCTTCCTGTAAT (SEQ ID NO: 13) |
| iSGR-F1 | CAGGAAGCATTGGTTTGGGTAT (SEQ ID NO: 14) |
| iSGR-R1 | GAACCTCATTAGAGCACGGCATT (SEQ ID NO: 15) |
| iSGR-R2 | TTAGTGGACCCCAACATTCTACC (SEQ ID NO: 16) |
| Tnt1-F | TCCTTGTTGGATTGGTAGCCAACTTTGTTG (SEQ ID |
| | NO: 17) |
| Tnt1-R | GCCAAAGCTTCACCCTCTAAAGCCT (SEQ ID NO: 18) |

C. Phylogeny Analysis, Protein Alignment, and Expression Pattern of MtSGR

SGR amino acid sequences from various plant species were directly downloaded from GenBank (www.ncbi.nlm.nih.gov) or retrieved via ORF Finder (www.ncbi.nlm.nih.gov/gorf/gorf.html). All sequences were subsequently aligned using MAFFT program and the G-INS-i strategy (align.bmr.kyushu-u.ac.jp/mafft/software/) (Katoh et al., 2009). The resulting alignment was edited with Genedoc software V2.6.02 (www.nrbsc.org/gfx/genedoc/) (Nicholas et al., 1997). A phylogenetic tree was built using Neighbor-Joining (NJ) method with a pairwise deletion option and 1000 replicate analyses by employing the MEGA 4.0 software (Kumar et al., 2008). In addition, phylogeny of these plant SGRs were reevaluated using Maximum Likelihood method with JTT model and 100 replicates (Guindon and Gascuel, 2003). Both trees showed complete agreement to each other, and bootstrap support values were printed on each branch respectively.

The coding sequence of MtSGR was used for the analysis of expression pattern based on M. truncatula Gene Expression Atlas through the BLAST function (bioinfo.noble.org/gene-atlas/v2).

D. Quantification of Chlorophyll and Photochemical Efficiency

First fully expanded leaves (−0.1 g) excised from 4- to 5-week-old plants were immediately frozen and ground in liquid N₂ quickly. Chlorophyll was extracted with 3 ml of 80% acetone containing 1 μM KOH (Schelbert et al., 2009). After centrifugation (10,000 g, 2 min), the supernatant was quantified using a spectrophotometer (Amon, 1949).

Maximum quantum yield of PSII (Fᵥ/Fₘ) was measured using a photosynthesis system LI-COR 6400 (Li-Cor, Lincoln, Nebr., USA) with a CO₂ concentration of 380 μmol/mol and flow rate of 400 μmol/s (Jiang et al., 2007).

E. Chloroplast Observation by Transmission Electron Microscope and Confocal Microscope

Leaves (5 mm×5 mm) were fixed in 3% (v/v) glutaraldehyde (Electron Microscopy Sciences, Hartfield, Pa., USA) in 1×PBS buffer (pH 7.0) overnight at 4° C. The samples were washed with 1×PBS and post-fixed in buffered 1% (v/v) Osmium tetroxide (Electron Microscopy Sciences, Hartfield, Pa., USA) for 2 hrs at 0° C. Then all specimens were washed, dehydrated in a series of ethanol, and embedded in LR white resin (London Resin Co. Ltd., London, UK). The resin was polymerized at 55° C. for 3 days. Ultra-thin sections were cut with a diamond knife on a MT-X ultramicrotome (Boeckeler Instruments, Inc., AZ, USA). Ultra-thin sections were put on formvar-carbon coated copper grids and stained with 2% uranyl acetate for 30 min followed by staining with Sato's lead for 2 min. The specimens were observed under a transmission electron microscope operated at 80 kV (JEOL 2000FX, JEOL, Tokyo, Japan).

Glutaraldehyde-fixed leaves were observed under Leica TCS SP2 AOBS Confocal Laser Scanning Microscope (Leica Microsystems, IL, USA) equipped with 63×HCX PL APO water immersion objective lens (numerical aperture 1.20). Auto-fluorescence was observed by exciting chloroplasts with 476 nm Argon laser and captured at 550-650 nm.

F. Construction of pANDA35HK-SGR Vector and Alfalfa Transformation

A 543 bp fragment of SGR (SEQ ID NO:5) was PCR-amplified from alfalfa using primers MsSGR-F and MsSGR-R (Table 5). The fragment was inserted into pENTR™/D-TOPO® cloning vector (Invitrogen, Chicago, Ill.) and transferred into the pANDA35HK vector by LR recombination reactions (Invitrogen, Chicago, Ill.). The final binary vector pANDA35HK-SGR was transferred into Agrobacterium tumefaciens strain EHA105 using the freezing/heat shock method. Transgenic alfalfa plants were obtained by Agrobacterium-mediated transformation (Austin et al., 1995). Alfalfa lines transformed with the original pANDA35HK vector were used as empty vector control.

G. RNA Extraction and qRT-PCR Analysis

Total RNA was extracted from transgenic alfalfa leaves using Trizol Reagent (Invitrogen) followed by chloroform extraction, isopropanol precipitation, and quantification by NanoDrop Spectrophotometer (ND-1000). After treatment with Turbo Dnase I (Ambion, Austin, Tex.), 1 μg RNA was reverse transcribed with SuperscriptIII (Invitrogen). The cDNA was diluted 1:20 and subsequently used as a template for qRT-PCR. The 10 μl reaction mix included 2 μl primers (0.5 μM of each primer), 5 μl Power Sybr (Applied Biosystems, Foster City, Calif.), 2 μl diluted cDNA, and 1 μl water. The primers used for qRT-PCR are listed in Table 5. RT-PCR data were analyzed with SDS 2.2.1 software (Applied Biosystem, Foster City, Calif.). PCR efficiency (E) was estimated using the LinRegPCR software (Ramakers et al., 2003), and the transcript levels were determined by relative quantification (Pfaffl, 2001) using the M. truncatula Actin gene (TC107326) as a reference.

H. Microarray Analysis

The whole plants of NF2089 mutant and wild-type (ecotypes R108) were kept in darkness for 5 days, then RNA was isolated from the fully expanded leaves of both NF2089 mutant and wild-type. Probe labeling, hybridization and scanning for microarray analysis were conducted according to the manufacturer's instructions (Affymetrix, www.affymetrix.com). Functional analysis of differentially expressed genes from microarray data was further analyzed using the GOEAST program by default adjusts the raw P values into false discovery rate using the Benjamini-Yekutieli method (Zheng and Wang, 2008) (omicslab.genetics.ac.cn/GOEAST/index.php). The enriched GO terms among differentially expressed genes (hypergeometric test and FDR less than 0.1) in biological process are shown as boxes in FIGS. 5A and 5B. The enriched GO annotation results were then classified using WEGO (Ye et al., 2006).

### I. Southern Blot Analysis

Genomic DNA was extracted from each transgenic line and wild-type using CTAB method and then treated with RNase (Qiagen, Carol Stream, Ill.). A 50 ng aliquot of plasmid DNA and 15 μg purified DNA of each line were digested with restriction endonuclease KpnI, electrophoresed on 0.8% agarose gels, and transferred to positively charged nylon membrane (Roche, Indianapolis, Ind.) by alkaline capillary blotting. The hybridization probe (gus) was labeled with digoxigenin (DIG) by PCR, and hybridization were carried out using DIG Luminescent Detection kit (Roche, Indianapolis, Ind.).

J. Forage Analysis of Transgenic Alfalfa Lines

Transgenic and control alfalfa were harvested at the early bud stage. The samples were dried at 50° C. for 72 hrs and ground through a Thomas-Wiley Laboratory Mill (Lehman Scientific, Wrightsville, Pa.) with 1 mm sieve. Near infrared reflectance spectroscopy (NIRS) was performed using Foss NIRS 6500 monochromator with a scanning range of 1100-2500 nm (Foss NIR Systems Inc., Silver Spring, Md.). Each sample was scanned eight times, and the average spectra were used for calibration. Mathematical and statistical treatments of all spectra were performed with WinISI™ III calibration development software (Foss NIR Systems Inc., Silver Spring, Md.). The existing commercial NIRS prediction equations (07AHY50) developed by NIRS Forage and Feed Testing Consortium were employed to calculate quality characteristics of alfalfa. The precision of NIRS has been assessed by regression analysis of the predicted values and actual determined values.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,461,648; U.S. Pat. No. 4,535,060; U.S. Pat. No. 5,000,000; U.S. Pat. No. 5,037,663; U.S. Pat. No. 5,302,523; U.S. Pat. No. 5,322,783; U.S. Pat. No. 5,384,253; U.S. Pat. No. 5,464,765; U.S. Pat. No. 5,508,184; U.S. Pat. No. 5,538,877; U.S. Pat. No. 5,538,880; U.S. Pat. No. 5,545,818; U.S. Pat. No. 5,550,318; U.S. Pat. No. 5,563,055; U.S. Pat. No. 5,591,616; U.S. Pat. No. 5,610,042;
U.S. Pat. Publ 20040049802
Barkoulas et al., Nat Genet., 2008.
Benedito et al., Plant J., 55:504-513, 2008.
Berger et al., Development, 136:823-832, 2009.
Bharathan et al., Science, 296:1858-1860, 2002.
Blein et al., Science, 322:1835-1839, 2008.
Bouchez et al., EMBO Journal, 8(13):4197-4204, 1989.
Bouton, Curr. Opin. Genetics and Develop., 17:553-558, 2007.
Bower et al., Plant Journal, 2:409-416. 1992.
Bromweld et al., Crop Sci., 20:254-255, 1980.
Buising et al., Mol Gen Genet, 243 (1):71-81. 1994.
Callis et al., Genes Dev., 1:1183-1200, 1987.
Casa et al., Proc. Natl. Acad. Sci. USA, 90 (23):11212-11216, 1993.
Champagne et al., Plant Cell, 19:3369-3378, 2007.
Chandler et al., The Plant Cell, 1:1175-1183, 1989.
Chuck et al., Plant Cell, 8:1277-1289, 1996.
Coen et al., Cell, 63:1311-1322, 1990.
Conkling et al., Plant Physiol., 93:1203-1211, 1990.
David-Schwartz et al., Plant Cell, 21:3093-3104, 2009.
D'Erfurth et al., Plant J., 34:95-106, 2003.
Di Giacomo et al., Plant Mol. Biol., 67:135-150, 2008.
Downward, BMJ, 328 (7450):1245-1248, 2004.
Ebert et al., Proc. Natl. Acad. Sci., 84:5745-5749, 1987.
Ellis et al., EMBO Journal, 6 (11):3203-3208, 1987.
Englbrecht et al., BMC Genomics, 5:39, 2004.
EP Patent No. 154204
Fire et al., Nature, 391: 806-11, 1998.
Fraley et al., Bio/Technology, 3:629-635, 1985.
Fromm et al., Nature, 319:791-793, 1986.
Gallie et al., The Plant Cell, 1:301-311, 1989.
Goellner et al., Mol. Plant Pathol., 11:169-77, 2010.
Graham et al., Plant Physiol., 131:872-877, 2003.
Gustafson et al., Crop Science, 43:755-759, 2003.
Hamilton et al., Proc. Natl. Acad. Sci. USA, 93 (18):9975-9979, 1996.
Hareven et al., Cell, 84:735-744, 1996.
Hartwig, Crop Sci., 23:237-239, 1983.
Hartwig, Crop Sci., 26:1135-1136, 1986.
Haseloff et al., Proc. Natl. Acad. Sci. USA, 94 (6):2122-2127, 1997.
Hay et al., Curr. Opin. Plant Biol., 12:593-598, 2009.
Heath, Curr. Opin. Plant Biol., 3:315-319, 2000.
Hensgens et al., Plant Mol. Biol., 22 (6):1101-1127, 1993.
Hinchee et al., Bio/Technol., 6:915-922, 1988.
Hofer et al., Plant Mol. Biol., 45:387-398, 2001.
Hofer et al., Curr. Biol., 7:581-587, 1997.
Hudspeth et al., Plant Mol. Biol., 12:579-589, 1989.
Janssen et al., Plant Physiol., 117:771-786, 1998.
Kaeppler et al., Plant Cell Reports, 9:415-418, 1990.
Knittel et al., Plant Cell Reports, 14 (2-3):81-86, 1994.
Koenig et al., Development, 136:2997-3006, 2009.
Kolattukudy et al., PNAS, 92:4080-4087, 1995.
Lawton et al., Plant Mol. Biol. 9:315-324, 1987.
Laxmi et al., PLoS One 3,:e1510, 2008.
Lehner et al., Brief Funct Genomic Proteomic, Apr. 3 (1):68-83, 2004.
Lincoln et al., Plant Cell, 6:1859-1876, 1994.
Long et al., Nature, 379:66-69, 1996.
Lynch et al., Plant Dis., 90:970, 2006.
McCabe et al, Bio-Technology, 11 (5):596-598, 1993.
McLean et al., Aust. J. Agric. Res., 31:951-956, 1980.
Miles et al., Plant Management Network, 2006.
Mysore et al., Trends Plant Sci., 9:97-104, 2004.
Odell et al., Nature, 313:810-812, 1985.
Ohta et al., Plant Cell, 13:1959-1968, 2001.
Omirulleh et al., Plant Mol. Biol., 21 (3):415-428, 1993.
Ow et al., Science, 234:856-859, 1986.
Parnis et al., Plant Cell, 9:2143-2158, 1997.
PCT App. WO 92/17598
PCT App. WO 94/09699
PCT App. WO 95/06128
PCT App. WO 97/4103
PCT App. WO 97/41228
Posada-Buitrago et al., Fungal Genet. Biol., 42:949-962, 2005.
Potrykus et al., Mol. Gen. Genet., 199:183-188, 1985.
Ramirez et al., Plant Physiol., 151:1878-1888, 2009.
Reichel et al., Proc. Natl. Acad. Sci., 93:5888-5893, 1996.
Ritala et al., Plant Mol. Biol., 24 (2):317-325, 1994.
Rogers et al., Methods Enzymol., 153:253-277, 1987.
Sambrook et al., In: Molecular Cloning-A Laboratory Manual (second edition), Cold Spring Harbour Laboratory Press, 1989.
Scholte et al., Mol. Breeding, 10:203-215, 2002.
Shani et al., Plant Cell, 21:3078-3092, 2009.
Sheen et al., Plant Journal, 8 (5):777-784, 1995.
Singsit et al., Transgenic Res., 6 (2):169-176, 1997.
Sinha et al., Genes Dev., 7:787-795, 1993.
Somleva et al., Crop Science, 42:2080-2087, 2002.
Stalker et al., Science, 242:419-422, 1988.
Sullivan et al., Mol. Gen. Genet., 215 (3):431-440, 1989.
Tadege et al., Plant J., 54:335-347, 2008.
Tadepally et al., BMC Evol. Biol., 8:176, 2008.
Takatsuji, Cell Mol. Life Sci., 54:582-596, 1998.
Thillet et al., J. Biol. Chem., 263:12500-12508, 1988.
Tian, Plant Cell Rep., 16:267-271, 1997.
Tomes et al., Plant. Mol. Biol., 14 (2):261-268, 1990.
Torbet et al., Crop Science, 38 (1):226-231, 1998.
Torbet et al., Plant Cell Reports, 14 (10):635-640, 1995.
Uppalapati et al., Molecular Plant Microbe Intera., 22:7-17, 2009.
Uppalapati et al., Studies on Host-Pathogen Interactions between Porphyra spp. (Rhodophyta, Bangiales) and Pythium porphyrae (Oomycota, Peronosporales):104, 1999.
Van de Mortel et al., Mol. Plant Microbe Interact., 20:887-99, 2007.
Van Eck et al., Plant Cell Reports, 14 (5):299-304, 1995
Vasil et al., Plant Physiol., 91:1575-1579, 1989.
Walker et al., Proc. Natl. Acad. Sci. USA, 84:6624-6628, 1987.
Wang et al., Molecular and Cellular Biology, 12 (8):3399-3406, 1992.
Wang et al., Plant Physiol., 146:1759-1772, 2008.
Yang et al., Proc. Natl. Acad. Sci. USA, 87:4144-4148, 1990.
Young et al., Curr. Opin. Plant Biol., 12:193-201, 2009.

What is claimed is:

1. A forage crop plant comprising down-regulated SGR gene function, wherein the plant exhibits an enhanced agronomic property as a result of said down-regulated SGR gene function.

2. The plant of claim 1, wherein the plant comprises a DNA molecule capable of expressing a nucleic acid sequence complementary to all or a portion of a SGR messenger RNA (mRNA).

3. The plant of claim 2, wherein the plant comprises a DNA molecule complementary to all or a portion of a SGR mRNA, wherein the DNA molecule down-regulates the function of the SGR gene relative to a plant lacking said DNA molecule.

4. The plant of claim 3, wherein said DNA molecule comprises SEQ ID NO:5.

5. The plant of claim 1, wherein the plant comprises a mutation in said SGR gene relative to a wild-type plant of the same species.

6. The plant of claim 5, wherein the mutated genomic SGR gene comprises a deletion, a point mutation or an insertion in said SGR gene.

7. The plant of claim 5, wherein the mutated genomic SGR gene is produced by irradiation, T-DNA insertion, transposon insertion or chemical mutagenesis.

8. The plant of claim 1, wherein the plant is alfalfa.

9. The plant of claim 1, wherein the enhanced agronomic property is selected from the group consisting of increased chlorophyll content, increased forage nutritional content, increased yield, and improved visual appearance compared to a plant in which the SGR gene is not down-regulated.

10. The plant of claim 3, wherein the DNA molecule is operably linked to a developmentally-regulated, organelle-specific, inducible, tissue-specific, constitutive, cell-specific, seed specific, or germination-specific promoter.

11. The plant of claim 3, further defined as an R0 transgenic plant.

12. The plant of claim 3, further defined as a progeny plant of any generation of an R0 transgenic plant, wherein the transgenic plant has inherited said DNA molecule.

13. The plant of claim 1, wherein the SGR gene encodes a polypeptide comprising a sequence selected from the group consisting of SEQ ID NO:2; SEQ ID NO:4; SEQ ID NO:20; SEQ ID NO:22; SEQ ID NO:24; SEQ ID NO:26; SEQ ID NO:28; SEQ ID NO:30; SEQ ID NO:32; SEQ ID NO:34; SEQ ID NO:36; SEQ ID NO:37; SEQ ID NO:38; SEQ ID NO:39; SEQ ID NO:40; and SEQ ID NO:41.

14. A seed that produces the plant of claim 1.

15. A DNA-containing plant part of the plant of claim 1.

16. The plant part of claim 15, further defined a protoplast, cell, meristem, root, leaf, node, pistil, anther, flower, seed, embryo, stalk or petiole.

17. A nucleic acid molecule comprising a nucleic acid sequence selected from the group consisting of:
(a) a nucleic acid sequence that hybridizes to the sequence of SEQ ID NO:1; SEQ ID NO:3; SEQ ID NO:5; SEQ ID NO:19; SEQ ID NO:21; SEQ ID NO:23; SEQ ID NO:25; SEQ ID NO:27; SEQ ID NO:29; SEQ ID NO:31; SEQ ID NO:33; or SEQ ID NO:35 under conditions of 1×SSC and 65° C.;
(b) a nucleic acid comprising SEQ ID NO:1; SEQ ID NO:3; SEQ ID NO:5; SEQ ID NO:19; SEQ ID NO:21; SEQ ID NO:23; SEQ ID NO:25; SEQ ID NO:27; SEQ ID NO:29; SEQ ID NO:31; SEQ ID NO:33; or SEQ ID NO:35 or a fragment thereof; and
(c) a nucleic acid sequence exhibiting at least 80% sequence identity to SEQ ID NO:1; SEQ ID NO:3; SEQ ID NO:5; SEQ ID NO:19; SEQ ID NO:21; SEQ ID NO:23; SEQ ID NO:25; SEQ ID NO:27; SEQ ID NO:29; SEQ ID NO:31; SEQ ID NO:33; or SEQ ID NO:35;
(d) a fragment of at least 19 contiguous nucleotides of a nucleic acid sequence of SEQ ID NO:1; SEQ ID NO:3; SEQ ID NO:5; SEQ ID NO:19; SEQ ID NO:21; SEQ ID NO:23; SEQ ID NO:25; SEQ ID NO:27; SEQ ID NO:29; SEQ ID NO:31; SEQ ID NO:33; or SEQ ID NO:35, or the reverse complement thereof, wherein the presence in a plant of a double stranded ribonucleotide sequence comprising at least one strand that is complementary to said fragment down-regulates SGR gene function in the plant;
wherein the nucleic acid sequence is operably linked to a heterologous promoter sequence.

18. The nucleic acid molecule of claim 17, wherein the DNA molecule comprises a nucleic acid sequence exhibiting at least 85%, 90%, 95%, 96%, 97%, 98% or 99% sequence identity to SEQ ID NO:1; SEQ ID NO:3; SEQ ID NO:5; SEQ ID NO:19; SEQ ID NO:21; SEQ ID NO:23; SEQ ID NO:25; SEQ ID NO:27; SEQ ID NO:29; SEQ ID NO:31; SEQ ID NO:33; or SEQ ID NO:35.

19. The nucleic acid molecule of claim 17, wherein the heterologous promoter sequence is a developmentally-regulated, organelle-specific, inducible, tissue-specific, constitutive, cell-specific, seed specific, or germination-specific promoter.

20. A transgenic plant cell comprising the nucleic acid molecule of claim 17.

21. A transgenic plant or plant part comprising the nucleic acid molecule of claim 17.

22. A method for producing forage comprising obtaining a plant according to claim 1 and collecting biomass for forage.

23. A method of conferring at least a first altered agronomic property to a plant comprising down-regulating SGR gene function in said plant relative to a plant in which SGR gene function is not down-regulated.

24. The method of claim 23, wherein the altered agronomic property is selected from the group consisting of increased nutritional content, increased chlorophyll content, increased yield, and improved visual appearance.


SEQUENCE LISTING (41 sequences)

SEQ ID NO: 1 - DNA, 1735 bp, Medicago truncatula
atgggtactctaaccaccgctcctcctcctatgctcacttctaagttcaaaccttctttttcacctcaacataaacctctttttccaaatagaagacggttatggaagaagaaccaatcaattgttcctgtatgtttttctttcttttttcttctcaatttttcaaataaatttagttttatttaatttttgactttgtatttgtgggtttggtttggtttggattttggaacaggttgctaggttatttggaccggctatatttgaagcatcaaaattgaaggttttgttcttaggaattgatgaagacaaacatccaggaaatcttccaaggacttatacgttaacacatagtgatgtaacctcaaaactcactttggcaatttctcaaaccattaataactctcaggtatttctcataattttgattatcaacatcatcatcacagtcatgtcttccctaatcatttgatggaagtcacaaaacagagttgaagaggttcacacacacaagaaaataatgaatattgaattttaggaataaaaaagtatacaaacatgggaattcaattgcagttgcagggatggtataatagattgcaaagggatgaagttgtggcgcagtggaagaaggtgaagggaaagatgtctctccatgttcattgtcatattagtggtggccattttttgttagatatatttgctagactaagatatttcatcttctgcaaagagttacccgtggtaagcatgtcttttttgactaaaatttttatctacgaaactctcacacacacacacacttaatgcatagaagaggggtagctcgagtctgaactcgagctccatggataaatatggggtattacaagttcgaactcgatttccaagggttaatatggggtattacaagttcgaattcactagagatatgtctccacacttattttactttttctataagatccgaaatattgttgtcttgacgatcattttctgagttttgccacatactaagggacaattacaaagattcttaaaactcattttaatcgagggtttttagagttcgatctcacacttcagcgtgtgttttcacctcaattgatagtaagttttaagcatcttcgtactaatctaagtttgaatttcaaatatatgacaaaaacataactcgtgaatgaattggtcaacaagttatcatggggtgtgagaactactatgtatgagtagttggccaactttttatcatttcaattgtgcttatgggacatgacaaacatgattaagactcggatacctaagttattcatcctaaaaaaatgaatcttagtgtcacttagacaaaaaataaaataaaattaagactatatcttattgtgattattaatattgtgtgtttgatttgatttaggtattgaaggcttttgtacacggtgacggcaatttattcaacaactatccggaattacaggaagcattggtttgggtatattttcattcaaagattccagaattcaacaaggtagaatgttggggtccactaaaggaggcttcacaacctactagtgggacccaaagggaccgccaaaatttgaccctacctcagccatgtcaagaaacttgcgagtgctgctttccaccgttgaagttgagcccaatgccgtgctctaatgaggttcacaatgatacttatgaacctattgatggaattgaaactcaacaatcactgtaa

SEQ ID NO: 2 - protein, 263 aa, Medicago truncatula
MGTLTTAPPPMLTSKFKPSFSPQHKPLFPNRRRLWKKNQSIVPVARLFGPAIFEASKLKVLFLGIDEDKHPGNLPRTYTLTHSDVTSKLTLAISQTINNSQLQGWYNRLQRDEVVAQWKKVKGKMSLHVHCHISGGHFLLDIFARLRYFIFCKELPVVLKAFVHGDGNLFNNYPELQEALVWVYFHSKIPEFNKVECWGPLKEASQPTSGTQRDRQNLTLPQPCQETCECCFPPLKLSPMPCSNEVHNDTYEPIDGIETQQSL

SEQ ID NO: 3 - DNA, 792 bp, Medicago sativa
atgggtactctaaccaccgctcctcctcctatgctcacttctaagttcaaaccttccttttcacctcaacataaacctctttttccaaatagaagacggttatggaagaagaaccaatcaattgttcctgttgctaggttatttggaccagctatatttgaagcatcgaaattgaaggttttgttcttaggaattgatgaagacaaacatccaggaaatcttccaaggacttatacgttaacacatagtgatgtaacctcaaaactcactttggcaatttctcaaaccattaataactctcagttgcagggatggtataatagattgcaaagggatgaagttgtggcgcagtggaagaaggtgaagggaaagatgtctctccatgttcattgtcacattagtggtggccattttttgttggatatattcgctagattaagatatttcatcttctgcaaagagttacccgtggtattgaaggcttttgtacacggtgacggcaatttattcaccaactatcctgaattacaggaagcattggtttgggtatattttcattcaaagattccagaattcaagaaggtagaatgttggggtccactaaaggaggcttcccaacctactagtgggacccaaagggaccaccaaattttggccctacctcagccatgtcaagaaaattgcgagtgctgctttccaccgttgaagttgagcccaatgccgtgctctaatgaggttcacaatgatacttatgaacctattgatggaattgaaactcaacaatcactgtaa

SEQ ID NO: 4 - protein, 263 aa, Medicago sativa
MGTLTTAPPPMLTSKFKPSFSPQHKPLFPNRRRLWKKNQSIVPVARLFGPAIFEASKLKVLFLGIDEDKHPGNLPRTYTLTHSDVTSKLTLAISQTINNSQLQGWYNRLQRDEVVAQWKKVKGKMSLHVHCHISGGHFLLDIFARLRYFIFCKELPVVLKAFVHGDGNLFTNYPELQEALVWVYFHSKIPEFKKVECWGPLKEASQPTSGTQRDHQILALPQPCQENCECCFPPLKLSPMPCSNEVHNDTYEPIDGIETQQSL

SEQ ID NO: 5 - DNA, 543 bp, Medicago sativa
actctaaccaccgctcctcctcctatgctcacttctaagttcaaaccttccttttcacctcaacataaacctctttttccaaatagaagacggttatggaagaagaaccaatcaattgttcctgttgctaggttatttggaccagctatatttgaagcatcgaaattgaaggttttgttcttaggaattgatgaagacaaacatccaggaaatcttccaaggacttatacgttaacacatagtgatgtaacctcaaaactcactttggcaatttctcaaaccattaataactctcagttgcagggatggtataatagattgcaaagggatgaagttgtggcgcagtggaagaaggtgaagggaaagatgtctctccatgttcattgtcacattagtggtggccattttttgttggatatattcgctagattaagatatttcatcttctgcaaagagttacccgtggtattgaaggcttttgtacacggtgacggcaatttattcaccaactatcctgaattacaggaagcattggtttgggta

SEQ ID NO: 6 - DNA, 21 bp, Artificial sequence
  PCR Primer
tccgccaccgttggattagag

SEQ ID NO: 7 - DNA, 21 bp, Artificial sequence
  PCR primer
ttggattttcccacgatgagc

SEQ ID NO: 8 - DNA, 18 bp, Artificial sequence
  PCR primer
actctaaccaccgctcct

SEQ ID NO: 9 - DNA, 20 bp, Artificial sequence
  PCR primer
tacccaaaccaatgcttcct

SEQ ID NO: 10 - DNA, 22 bp, Artificial sequence
  PCR primer
atgggtactctaaccaccgctc

SEQ ID NO: 11 - DNA, 27 bp, Artificial sequence
  PCR primer
ttacagtgattgttgagtttcaattcc

SEQ ID NO: 12 - DNA, 22 bp, Artificial sequence
  PCR primer
caccactctaaccaccgctcct

SEQ ID NO: 13 - DNA, 25 bp, Artificial sequence
  PCR primer
tacccaaaccaatgcttcctgtaat

SEQ ID NO: 14 - DNA, 22 bp, Artificial sequence
  PCR primer
caggaagcattggtttgggtat

SEQ ID NO: 15 - DNA, 23 bp, Artificial sequence
  PCR primer
gaacctcattagagcacggcatt

SEQ ID NO: 16 - DNA, 23 bp, Artificial sequence
  PCR primer
ttagtggaccccaacattctacc

SEQ ID NO: 17 - DNA, 30 bp, Artificial sequence
  PCR primer
tccttgttggattggtagccaactttgttg

SEQ ID NO: 18 - DNA, 25 bp, Artificial sequence
  PCR primer
gccaaagcttcaccctctaaagcct

SEQ ID NO: 19 - DNA, 1194 bp, Arabidopsis thaliana
atgtgtagtttgtcggcgattatgttgttaccaacgaagctgaaaccagcttattcagacaaacggagtaacagtagcagcagcagctcactcttcttcaacaatagaagatccaagaagaagaaccaatcgattgttcccgtaaggatttgttctcttaatcattttgacatatctttgttttatgtgtctcagttatggatatatctatgcctctgtctttattgatggaacttgaaacttttgggatcggtgcaggttgcaaggttgtttggaccggcgattttcgaatcatccaaattgaaagtactcttcttaggggttgatgagaagaagcatccttcaacgctccctaggacttacacactcactcacagtgacattacagctaaactaaccttagctatttctcaatccataaacaactctcaggtaaaaacacctctctctctggtcttaacatatctacatggctctgtttataggagtagaatgtttatgtatttgattatttggagcaaatattagaggcttatgtatgattgttttaacctatgtatgtgtctatattcaccagaatctcttaacactttagaggctgatatgtatagaaatgttttttatagttgcaaggatgggcaaataggctataccgggatgaagttgtggcagaatggaagaaagtgaaagggaaaatgtcgcttcacgttcattgtcacataagcggtggccatttccttttagatctctttgcaaagtttcgatatttcatcttttgcaaagaactacctgtggtaagttcccaaattcataagaaactttttattggtttcatgtaaaacaaaatgtttattgttttgatatatacaggtgttgaaggcttttgtgcatggagatgggaacttgttgaacaactatcctgagctacaagaagctcttgtttgggtctatttccattctaatgtcaatgagttcaacaaagtcgagtgttggggtccgctttgggaagctgtttcgcctgatggtcacaagactgagactcttcccgaggctcggtgtgcggacgagtgtagttgttgttttccaaccgttagctcgattccatggtctcatagtcttagtaatgaaggtgtaaatggttactctgggactcagactgagggaattgctactccaaatccggagaaactctag

SEQ ID NO: 20 - protein, 268 aa, Arabidopsis thaliana
MCSLSAIMLLPTKLKPAYSDKRSNSSSSSSLFFNNRRSKKKNQSIVPVARLFGPAIFESSKLKVLFLGVDEKKHPSTLPRTYTLTHSDITAKLTLAISQSINNSQLQGWANRLYRDEVVAEWKKVKGKMSLHVHCHISGGHFLLDLFAKFRYFIFCKELPVVLKAFVHGDGNLLNNYPELQEALVWVYFHSNVNEFNKVECWGPLWEAVSPDGHKTETLPEARCADECSCCFPTVSSIPWSHSLSNEGVNGYSGTQTEGIATPNPEKL

SEQ ID NO: 21 - DNA, 1405 bp, Arabidopsis thaliana
atgtgtagtttggctacaaatctgttactaccatcgaagatgaaaccagtttttccagagaaactgagcactagctcactctgtgtcaccactagaagatctaagatgaagaaccgatctattgttcctgtaagattctcaaatctttttcctctacactctgttttcaaagttactatatttgttaataatttcattacttgtttgatttatgttcgtttccatgaatatctgttactatttctggaaattattagaaacaataataagttccattactttagtttgtatactctgtttttagggtttgtctctatgttgcgccttgtttgaatctgaaatttgatggatcatattaaaggtttgatatttctttgttttgaatggatataaaggttgcaagattgtttggaccggcgatttttgaagcctccaaattgaaagtgttattcttaggagttgatgagaagaagcatccagcaaaacttccaagaacttacactcttactcacagtgacataaccgctaaattaactttagctatatctcaatccattaataactctcaggtaaaattataaaccgtacagttgcaacgtttaggatttagggttgcgtaaaagtgatgtaacgttttcggttcttgctttcaccagttgcaaggatgggcaaataaattgttccgggacgaagtagtgggcgagtggaagaaagtgaaaggtaaaatgtcgcttcatgttcattgccacattagcggaggccacttcttcttgaatctcatcgcgaagcttcggtactacatcttttgcaaagaattacctgtggtaagttacttttgtgcaagcttttttgagtaatagtctagtggtttattgtcatgttaattaggaaatatattacaaaaatatattgtagataatttttaacttgtatggagcaagcatagactccaagatgtctgtctggaattcttggctaataacagatatatttatattgtatatgatatgtaatagtaataccacgattgattgagtatattggacctattgatttataggtactggaagcttttgcccatggagatgagtatttgttaaataatcaccccgagctacaagaatctcctgtttgggtttatttccattccaacatcccggagtacaacaaggtcgaatgttggggaccgctttgggaggccatgtcgcagcaccagcacgacggaaggacccacaagaagagtgaaactctaccggagctaccttgtcctgatgagtgcaagtgttgctttccgacggttagcacgattccgtggtctcatcgtcattatcaacataccgcagcggatgagaatgttgcggatggcctgttggaaatacctaaccctgggaaatcaaagggatag

SEQ ID NO: 22 - protein, 271 aa, Arabidopsis thaliana
MCSLATNLLLPSKMKPVFPEKLSTSSLCVTTRRSKMKNRSIVPVARLFGPAIFEASKLKVLFLGVDEKKHPAKLPRTYTLTHSDITAKLTLAISQSINNSQLQGWANKLFRDEVVGEWKKVKGKMSLHVHCHISGGHFFLNLIAKLRYYIFCKELPVVLEAFAHGDEYLLNNHPELQESPVWVYFHSNIPEYNKVECWGPLWEAMSQHQHDGRTHKKSETLPELPCPDECKCCFPTVSTIPWSHRHYQHTAADENVADGLLEIPNPGKSKG

SEQ ID NO: 23 - DNA, 1004 bp, Glycine max
acttgaataaaagctaaataaatattaaagcctttccttcatcaaggtactgtttgatctttgaatttcagtcctcaaagattggaacaacaggaacatgtgtactctcacaactgttcctgtgctcccttctaagcttaacaagccttcgctttctccgcaccacaattctctttttccctactgtggaagacgggtcgggaagaagaacaaagcaatggttcctgttgcaaggttgttcgggccagccatatttgaagcctcaaaactgaaggttttgttcttaggagtggacgaaaataagcacccaggaaatctcccaaggacttatacgctaacccatagtgatataaccgctaagctcaccttggcaatctctcaaaccataaataattctcagctgcaggggtggtacaacagatttcaaagggacgaagtggtggcacagtggaaaaaggtgaagggaaggatgtctctgcacgttcactgccacattagtggaggtcattttctcttggatatattagcaaggttaagatacttcatcttctgcaaggagctaccagtggtgttgaaggccgtcgttcacggcgatgaaaacctattcaacagctacccagaattgcaagatgccttggtttgggtctactttcactcaaacattccagaattcaacaaggtggaatgttggggcccactgaaggaagcgtcagcacccacaggtggggtccaggaggaggggttggcaattccacagccatgccaagaagaatgccaatgttgctttccaccgcttacgttgagccctattcagtggtctaaacaagttcccagccgccattacgaaccttgtgatgggattgggacccaacaaaatctataaacagccaaactactacaattcacagttacggggatgctgccaaattggcaaattccctgtatacaaattaaatattcgaaacccatttcaatgtaaataaggaagctcgtatttgcaattt

SEQ ID NO: 24 - protein, 261 aa, Glycine max
MCTLTTVPVLPSKLNKPSLSPHHNSLFPYCGRRVGKKNKAMVPVARLFGPAIFEASKLKVLFLGVDENKHPGNLPRTYTLTHSDITAKLTLAISQTINNSQLQGWYNRFQRDEVVAQWKKVKGRMSLHVHCHISGGHFLLDILARLRYFIFCKELPVVLKAVVHGDENLFNSYPELQDALVWVYFHSNIPEFNKVECWGPLKEASAPTGGVQEEGLAIPQPCQEECQCCFPPLTLSPIQWSKQVPSRHYEPCDGIGTQQNL

SEQ ID NO: 25 - DNA, 1081 bp, Glycine max
ttctcctcaagcaaaacctagagctcccagactcctccactacttagaatattaaaagccccttccttcatcaaggtactactgtttcatcttccctcaaagattggaacaacaacaggaacatgggtactctaacaactgttcctgtgctcccttctaagcttaacaagccttcgctttctccgcgtcacaattctctttttccctactacggaagacgcgtcgggaagaagaacaaagcaatggttcctgttgctaggttgttcgggccagccatatttgaagcctcaaagcttaaggttttattcttaggagtggacgaaaataaacacccaggaaatctcccaaggacttatactctaacccatagtgatataaccgctaagctcaccttggcaatctctcaaaccataaataattctcagttacaggggtggtacaacagattgcaaagggacgaagtggtggcacagtggaagaaggtgaagggaaagatgtctctgcacgtacactgccacatcagtggtggtcattttctcttagatatattagcaaggttacgatacttcatcttctgcaaggagctaccagtggtgttgaaggcggtggttcacggcgacgaaaacctattcaacaactacccagaattgcaagatgccttggtttgggtttactttcactcaaacattccagaattcaacaaggtggaatgttggggcccactgaaggaagcgtcagcaccaataggtggggccaaggaagagagtgagcaagaaactcttctaagtaaggagggcttggcaattccacagccatgccaagaggaatgcgaatgttgctttccaccgctgacgttaagcccaattcagtggtctcaacaagttcccagccaccattacgaaccttgtgatgggattgagacccaacaaagtctataaacttccaaactatcatacacagaaaagaaaaaaaataaaatcacatcacagttacagggatgctggcaaattcatatgccatttccctgtatacatcattaggacaaatcaaatattcaaaacccatttcaatgtaaagaagg

SEQ ID NO: 26 - protein, 271 aa, Glycine max
MGTLTTVPVLPSKLNKPSLSPRHNSLFPYYGRRVGKKNKAMVPVARLFGPAIFEASKLKVLFLGVDENKHPGNLPRTYTLTHSDITAKLTLAISQTINNSQLQGWYNRLQRDEVVAQWKKVKGKMSLHVHCHISGGHFLLDILARLRYFIFCKELPVVLKAVVHGDENLFNNYPELQDALVWVYFHSNIPEFNKVECWGPLKEASAPIGGAKEESEQETLLSKEGLAIPQPCQEECECCFPPLTLSPIQWSQQVPSHHYEPCDGIETQQSL

SEQ ID NO: 27 - DNA, 786 bp, Pisum sativum
atggatactctaacgagtgctcctttactcactactaagttcaaaccttcgttttctcctcaacagaaaccatgttttccgcatagaagacggtttgagaatgggaagaagaatcaatcaattgttcctgtagctaggttatttggaccagctatatttgaagcttcgaaacttaaggttttgttcttaggaattgacgaaaacaaacacccaggaaatcttccgaggacttatacgttaacacatagtgatgttacctcaaaactcactttggcaatttctcaaaccataaacaactctcagttgcagggatggtacaatagattgcaaagagatgaagtggttgcgcaatggaagaaggtgaagggaaagatgtctctacatgttcactgtcacattagtggaggccattttcttttggatatctttgctaggcttagatacttcatcttctgcaaagagttacctgtggtgttgaaggcttttgttcacggtgacggcaatttattcaacaattatccggaattagaggaatcattagtttgggtattttttcattcaaagattcgagaattcaacaaggtagaatgttggggtccactaaaggaggcttcacaacctactagtgggacccactctgatttgaaactgccccaatcttgtgaagaggattgtgagtgttgttttccaccgttgaatttaagcccaattccgtgctctaatgaagttattaataatacttatgaacctattgatggaattggaacccaacatggtaacttgtaa

SEQ ID NO: 28 - protein, 261 aa, Pisum sativum
MDTLTSAPLLTTKFKPSFSPQQKPCFPHRRRFENGKKNQSIVPVARLFGPAIFEASKLKVLFLGIDENKHPGNLPRTYTLTHSDVTSKLTLAISQTINNSQLQGWYNRLQRDEVVAQWKKVKGKMSLHVHCHISGGHFLLDIFARLRYFIFCKELPVVLKAFVHGDGNLFNNYPELEESLVWVFFHSKIREFNKVECWGPLKEASQPTSGTHSDLKLPQSCEEDCECCFPPLNLSPIPCSNEVINNTYEPIDGIGTQHGNL

SEQ ID NO: 29 - DNA, 995 bp, Capsicum annuum
cacaactcctctttaagtttctactcattctctttgccaactttgatcaaaaacaccttataataaattgatcaagattcaaggagttttgggtacccaatttcttgatgaaatggggactttgactgcttctctagtagctccatctaagctcaaccctgaaaagcatagctctctttttgtatacaaaactagaagaaagtcccacaagaatcaatccatagtccctgtggcaaggctatttggaccagctatatttgaagcatcaaagttgaaggtacttttcttgggagttgatgagaaaaagcacccaggaaagttgccaagaacatatacactgactcatagtgatattacttctaaactcactttggcaatctctcaaaccatcaataactctcagttgcaaggttggtataatagacttcaaagggatgaagtggttgcagaatggaagaaagttaaagggaagatgtcactccatgtacattgccacattagtggaggccattttatgttagacttatttgctagactcagatactatatcttctgcaaagaactccctgtggttctgaaggcttttgttcatggagatgagaatttactaaagaattatccagagttgcaacaagctttagtttgggtatattttcactcaaacattcaagaattcaacaaagtagaatgttggggcccactcaaagatgcagcctccccctcatcaagtggggtaggtgggggtatgaatacaagttttacaagcaatagcaacatcaagtggaatttaccaaagccttgtgaagagacttgtacatgttgctttcccccaatgagtgttatcccttggccttctactactaatgtggaaaatgggaccatacaacaaggcttgcaagagcagcaaagctgaaaaaagacagtaattctgttgctttattatgtgatttgatgtaattaattaattaattaattatcatcattatagggtttgt

SEQ ID NO: 30 - protein, 266 aa, Capsicum annuum
MGTLTASLVAPSKLNPEKHSSLFVYKTRRKSHKNQSIVPVARLFGPAIFEASKLKVLFLGVDEKKHPGKLPRTYTLTHSDITSKLTLAISQTINNSQLQGWYNRLQRDEVVAEWKKVKGKMSLHVHCHISGGHFMLDLFARLRYYIFCKELPVVLKAFVHGDENLLKNYPELQQALVWVYFHSNIQEFNKVECWGPLKDAASPSSSGVGGGMNTSFTSNSNIKWNLPKPCEETCTCCFPPMSVIPWPSTTNVENGTIQQGLQEQQS

SEQ ID NO: 31 - DNA, 1169 bp, Solanum lycopersicum
ggacttttatcaaacagctaacttgcaatttcttttatatacttttaacattcaacaagattgttttattactggaaatttccagtaatattggaactccaagattcaaggagttttgggtacccaatttcttgtagaaaaaatgggaactttgactacttctctagtggttccatctaagctcaacaatgaacaacagagctctatttttatacacaaaactagaaggaaatgcaagaagaatcaatccatagtacctgtggcaaggttatttggaccagctatatttgaagcttcaaaattgaaggtactttttttgggagttgatgaagaaaagcatccaggaaagttgccaagaacatatacactgactcatagtgatattacttctaaacttactttggctatctcccaaaccatcaataattctcagttgcaaggttggtataacagacttcaaagagatgaagttgttgcagagtggaagaaagtaaaagggaagatgtcacttcatgtccattgccacattagtggaggccattttatgttagacttatttgctagactcagaaactacatcttctgcaaagaactccctgtggttctcaaggcttttgttcatggagatgagaatttactaaggaattatccagagttacaagaagctttagtttgggtatattttcattcaaacattcaagaattcaacaaagtagaatgttggggtccactcagagatgcaacttccccctcatcttcttctggtggggtaggtggggtgaagagtacaagttttacaagcaatagcaacaaaaaatgggaattaccaaagccttgtgaagaggcttgtgcctgttgctttcccccagtgagtgttatgccttggctttcttcaaatcttgatggggtaggtgaggaaaatgggaccatccaacaaggcttgcaagagcagcaaagttgaaaaaaatgagatagtgattatgtttggtttattatgtgatttgatgtaattaactatttaattagttgtcattatagggtttgtggaagcagcattatttatttggttgaaatgtaaataagaattcagaggtcatgattaaagacaagaacatcaagtttaggatggtgatacataactatacaattattgtaagttgggttgtgcc

SEQ ID NO: 32 - protein, 271 aa, Solanum lycopersicum
MGTLTTSLVVPSKLNNEQQSSIFIHKTRRKCKKNQSIVPVARLFGPAIFEASKLKVLFLGVDEEKHPGKLPRTYTLTHSDITSKLTLAISQTINNSQLQGWYNRLQRDEVVAEWKKVKGKMSLHVHCHISGGHFMLDLFARLRNYIFCKELPVVLKAFVHGDENLLRNYPELQEALVWVYFHSNIQEFNKVECGPLRDATSPSSSSGGVGGVKSTSFTSNSNKKWELPKPCEEACACCFPPVSVMPWLSSNLDGVGEENGTIQQGLQEQQS

SEQ ID NO: 33 - DNA, 1524 bp, Oryza sativa
gagaagccaatccgcgaagctaccaaaccgagccaaaattttgatcgatcgatcggccggccggtgtctttgttgaggcgtgcaaggctcgtgctttcgccgagagggattaattagccgcgcctccagcatctccggcctccatagagatcccagcttataattaaagctgtcaagagtcgttttggttgttaggcttaaagttaactactgctggctagctaggactaagagatccgagggagcagacatggctgctgctacttcgaccatgtccctgcttcctcccatcacccagcagcagcggtggcacgccgccgactccctcgtcgtcctcgcctcccgctgccacaactctcgccgccgccgccgctgccgctacgtcgtgccgagggcgaggctgttcgggccggcgatcttcgaggcgtcgaagctgaaggtgctgttcctgggggtggacgaggagaagcaccagcacccggggaagctgccgcggacgtacacgctgacgcacagcgacgtgacggcgaggctgacgctggcggtgtcgcacaccatcaaccgggcgcagctgcaggggtggtacaacaagctgcagcgggacgaggtggtggcggagtggaagaaggtgcagggccacatgtcgctgcacgtccactgccacatctccggcggccacgtcctcctcgacctcatcgccggcctccgctactacatcttccgcaaggagctccccgtggttctgaaggcgttcgtccacggcgacggcaacctgttcagccggcacccggagctggaggaggccacggtgtgggtctacttccactccaacctcccacgcttcaaccgcgtcgagtgctggggcccgctccgcgacgccggagcgccgcccgaggaagacgacgccgtcgccgccgcggcggccgaggaggcggcggcggagcagatgcccgcggccggcgagtggccgcggcggtgcccggggcagtgcgactgctgcttcccgccatacagcctcatcccctggccgcaccagcacgacgtcgccgccgccgacggccagccgcagcagtgacggcggcgccgccgcgattcgtagattagcgaattcgtttggttgccatggtacacctacacgcatgcaatgtcgccaaatgacgctgcgtgctttgcgttaaattctttaattattcgtagtggcatttagctcagctcagctcagctctgcaattagcacatatttgattagctttagggaatgagtgtggtgagcttgtaaatagaagaacactagtagatggcaagcaaatggaaacgatcgatgatgcagtagatgttactgcaaaaatggcaattattagatgaagtcgacacacaaagacagatctcgtttcatttgtacattctacagtctacgtcgatgaggacatgactggatttgtatagcaaattagtactagcagctaataagtgtggattaattatgaaacacgattgtttaaaaaaaaaaaaaaaaaaaaaaaa

SEQ ID NO: 34 - protein, 274 aa, Oryza sativa
MAAATSTMSLLPPITQQQRWHAADSLVVLASRCHNSRRRRRCRYVVPRARLFGPAIFEASKLKVLFLGVDEEKHQHPGKLPRTYTLTHSDVTARLTLAVSHTINRAQLQGWYNKLQRDEVVAEWKKVQGHMSLHVHCHISGGHVLLDLIAGLRYYIFRKELPVVLKAFVHGDGNLFSRHPELEEATVWVYFHSNLPRFNRVECWGPLRDAGAPPEEDDAVAAAAAEEAAAEQMPAAGEWPRRCPGQCDCCFPPYSLIPWPHQHDVAAADGQPQQ

SEQ ID NO: 35 - DNA, 1867 bp, Sorghum bicolor
gcacgaggagacgaacgagagaaagcctgtgagcttgcccgcccgcgcacacaaccgcgacgtttcccgacccgtcactccaccaccaccaactcgccctgcacacacagtccttcctgcacacgcgccgcccggcggctgcccgtcacgcactcatataacgactccatacggtccagcaagccactcgccaggcagtcgggaagcgcgaccgcaccagctcctgtactacctgcagacgcccatcggccagaacactcgcgtagccaggctagtccaagattctctcagtccggtcctccatccggatctcgccgaggcgtgcaagattactgcttcgccgaggattaattagccgccggccgagcttctcctgagagtcggcagcttttctcggtcccgatttgccctctgctgctcgtggtgcgccgctctcctagggccctgcaggcgaagggaaggatccgaatcaacatggccgcagccactgccgccgccgcttctaccatgtccctgcccccgatctcccagctcaggcagcagcagcacggcgcgggcgccgtggtcgtgttccggcggcgggcccgggacgcgcggcggaggcgatacgtcgtgccgacggcgaggctgttcgggccggcgatcttcgaggcgtccaagctgaaggtgctgttcctgggcgtggacgaggagagcaacaacaagcacgggcacccgacgacgccgtcgccgacttccccgccgctgccgctactgccgcggacgtacacgctgacgcacagcgacgtgacggccagcctgacgctggccgtgtcccacaccatcaaccgcgcgcagctgcaagggtggtacaaccgcctgcagcgggacgaggtggtggcggagtggaagaaggtgcgcgggcggatgtcgctgcacgtgcactgccacatctccggcggccacttcctgctcgacctcatcgccggcctccgctactacatcttccgcaaggagctccccgtggtgctcaaggctttcgtgcacggcgacggcgacctgttcagccggcacccggagctggaggatgccccggtgtgggtctacttccactccaacctgacccgcttcaaccgcgtcgagtgctggggtccgctgcgcgacgccgccgcgccgccggccgaggacgactccaccgcgccggccgccgcctccaacaaggatgggcagatgccgcccgtgggcgagtggccgtaccggtgtccccagcagtgcgactgctgcttcccgccgcacagcctcatcccctggccgaacgagcgcgacatggcggccgccgccgccgatgcctcctccgccgccggccaggcccaacagcagtagtgaaactgcacgtacggagggggatcgagggaccttcgctgcaattgtgtaatgtaagccgtagctagactactagactagcgtgttgtttgcatttggttgggtagtagctgcaagcgcgcgccgtgcaggtgtgcagggggcgccaacacttgcatgccccgcgctattctttaattggtagcttagctgagctcagttcatttccaagttgtgttcatctttggaagtacagtagcttggttgaggtcggtgggatgaacatggtggtcttgtaaatagctagctagaacactttgatctggatggcaaacctgcaaagaatctgcacgtacatagtacacatgttgctgcaaagtggcaagtttaagtgtcggcaggcaaagtatcatccggtgtactacttaaattagaactatactatttcttgaactgtaattccccctggatttgtgtagtaaatggaatgtaaatgactaattatttaaaaaaaaaaaaaaaaaaa

SEQ ID NO: 36 - protein, 295 aa, Sorghum bicolor
MAAATAAAASTMSLPPISQLRQQQHGAGAVVVFRRRARDARRRRYVVPTARLFGPAIFEASKLKVLFLGVDEESNNKHGHPTTPSPTSPPLPLLPRTYTLTHSDVTASLTLAVSHTINRAQLQGWYNRLQRDEVVAEWKKVRGRMSLHVHCHISGGHFLLDLIAGLRYYIFRKELPVVLKAFVHGDGDLFSRHPELEDAPVWVYFHSNLTRFNRVECWGPLRDAAAPPAEDDSTAPAAASNKDGQMPPVGEWPYRCPQQCDCCFPPHSLIPWPNERDMAAAAADASSAAGQAQQQ

SEQ ID NO: 37 - protein, 267 aa, Nicotiana tabacum
MGTLTTSLAVPSKLNPEKQSSIFIYKTRRKSNKNQSIVPVARLFGPAIFEASKLKVLFLGVDEKKHPGKRPRTYTLTHSDVTSKLTLAISQTINNSQLQGWYNRLQRDEVVAEWKKVKGKMSLHVHCHISGGHFLLDLFARLRYYIFCKELPVVLKAFVHGDKNLLNNYPELQEALVWVYFHSNIQEFNKVECWGPLKEAASPSSSGGVGGKMRNTNTTSNSNWDLPQPCQESCSCCFPPMSLIPWPSDDISGTDGGPIQGLQEQQS

SEQ ID NO: 38 - protein, 184 aa, Zea mays
MAAAASTMSSRKGAGAVVVRRWDARRRRYVVTARGAASKKVGVDGSSKHHAHHAARTYTTHSDVTASTAVSHTNRAGWYNRRDVVAWKKVRGRMSHVHCHSGGHDAGRYYRKVVAVHGDGDSRHATVWVYHSNARNRVCWGRDAAAAADDSTAAAASAMGMVGWHRCCDCCHSWNDMAAAAGVR

SEQ ID NO: 39 - protein, 188 aa, Zea mays
MAAATAAASTMSSRHGAGAMRRRWVARRRRYVVTARGAASKKVGVDDAGSKHGRTYTTHSDVTARTAVSHTNRAGWYNRRDVVAWKKVRGRMSHVHCHSGGHDAGRYVRKVVKAVHGDGDSRHATVWVYHSNARNRVCWGRDAAAADDSTADASNSKAGMMAMCWHRCCGCCHSWNHDMAAADASGSA

SEQ ID NO: 40 - protein, 149 aa, Physcomitrella patens
MSLHVHCHISGGNFLHNLIANLRFYIFRKELPVVLEAFRHGDEELLKTYPDLDNAMVWVYFHSNVKEYNRVECWGPLVEAAKSASEEAKEAIHHAMEEMERQWPKQLWPGKVCDEPCECCSRHGTVIPLPKSFDLLQEEQKRQQEGSPS

SEQ ID NO: 41 - protein, 229 aa, Physcomitrella patens
MALQTARLFGPSIFEAAKLKVLLQGAAKKHPEELPRVYTLTHSDVTSKITLAISREINKAQLKGWYSKLQRDEVVAEWRKVQGKMSLHVHCHISGGNVLHNLIANLRFYIFRKELPVVLEAFRHGDEELLKEYPDLDNSLVWVYFHSNVNEYNRLECWGPLAEAAKGEDEAKEVIHHAMEEIEKKWPKQLWPGKTCEAPCDCCSRHGTLIPVPKTFELLQMQEEQKRST